(19) United States
(12) Patent Application Publication
(10) Pub. No.: US 2012/0027842 A1
(43) Pub. Date: Feb. 2, 2012

(54) TOPICAL FORMULATIONS OF FLAP INHIBITORS FOR ADMINISTRATION TO AN EYE

(75) Inventors: John Howard Hutchinson, San Diego, CA (US); Jillian F. Evans, Rancho Santa Fe, CA (US); Nicholas Simon Stock, San Diego, CA (US)

(73) Assignee: Amira Pharmaceuticals, Inc., San Diego, CA (US)

(21) Appl. No.: 13/141,673

(22) PCT Filed: Dec. 21, 2009
(86) PCT No.: PCT/US09/69066

§ 371 Date: Sep. 13, 2011

Related U.S. Application Data
(60) Provisional application No. 61/140,574, filed on Dec. 23, 2008.
(60) Provisional application No. 61/176,451, filed on May 7, 2009.

(51) Int. Cl. A61K 9/127; A61K 31/5377; A61K 38/13; A61K 31/56; C07D 403/14; C07D 401/14; C07D 413/14; A61P 31/12; A61P 31/10; A61P 31/00; A61P 29/00; A61P 35/00; A61P 27/02; A61P 27/06; A61P 27/04; A61K 31/513

(52) U.S. Cl. 424/450; 514/255.05; 514/269; 514/235.2; 514/20.5; 514/171; 544/295; 544/298; 544/123

(57) ABSTRACT

Described herein are topical formulations for administration to an eye, wherein the formulation is administered to treat ophthalmic diseases, disorders, or conditions. A topical formulation for administration to an eye disclosed herein comprises a therapeutically-effective amount of a FLAP inhibitor compound formulated for topical administration to the eye.

### RELATED APPLICATIONS

This application claims benefit of U.S. Provisional Application No. 61/140,574, entitled “TOPICAL FORMULATIONS OF FLAP INHIBITORS FOR ADMINISTRATION TO AN EYE” filed on Dec. 23, 2008, and U.S. Provisional Application No. 61/176,451, entitled “TOPICAL FORMULATIONS OF FLAP INHIBITORS FOR ADMINISTRATION TO AN EYE” filed on May 7, 2009, both of which are herein incorporated by reference.

### FIELD OF THE INVENTION

Described herein are topical formulations for administration to an eye of a mammal that include a 5-lipoxygenase-activating protein (FLAP) inhibitor compound and methods of use thereof in the treatment or prevention of ophthalmic diseases, disorders or conditions.

### BACKGROUND OF THE INVENTION

Ophthalmic diseases, disorders or conditions include any abnormal state of an eye and/or a related tissue. By way of non-limiting example, ophthalmic disorders include age-related macular degeneration, allergic conjunctivitis, anterior segment scarring, blepharitis, blepharoconjunctivitis, a bullous disorder, cicatricial pemphigoid, conjunctival melanoma, conjunctivitis, contact lens-associated giant papillary conjunctivitis, diabetic retinopathy, dry eye, episcleritis, glaucoma, gliosis, granuloma annulare, Graves' ophthalmopathy, intraocular melanoma, keratitis, keratoconjunctivitis, pain, pinguecula, post-surgical pain, proliferative vitreoretinopathy, pterygia, scarring, scleritis, Sjögren's syndrome, uveitis, vernal keratoconjunctivitis or combinations thereof.

### SUMMARY OF THE INVENTION

Described herein, in certain embodiments, are formulations for administration to an eye, wherein the formulation is administered to treat an ophthalmic disease, disorder or condition (i.e., an abnormal state of an eye and/or a related tissue). Described herein, in certain embodiments, are topical formulations for administration to an eye. The topical formulation is formulated with excipients that are ophthalmically acceptable.

In certain embodiments, the formulation is administered to treat an immune disorder (e.g. an autoimmune disorder); a proliferation disorder (e.g., intraocular melanoma); contact with an allergen, and/or an irritant; a fibroblast disorder (e.g., scarring); or combinations thereof. Described herein, in certain embodiments, are topical formulations for administration to an eye, wherein the formulation is administered to treat age-related macular degeneration, allergic conjunctivitis, anterior segment scarring, blepharitis, blepharoconjunctivitis, a bullous disorder, cicatricial pemphigoid, conjunctival melanoma, conjunctivitis, contact lens-associated giant papillary conjunctivitis, diabetic retinopathy, dry eye, episcleritis, glaucoma, gliosis, granuloma annulare, Graves' ophthalmopathy, intraocular melanoma, keratitis, keratoconjunctivitis, pain, pinguecula, post-surgical pain, proliferative vitreoretinopathy, pterygia, scarring, scleritis, Sjögren's syndrome, uveitis, vernal keratoconjunctivitis or combinations thereof. In some embodiments, a topical formulation for administration to an eye disclosed herein comprises a therapeutically-effective amount of a FLAP inhibitor. In some embodiments, a topical formulation for administration to an eye disclosed herein is administered before or after contact with an allergen and/or irritant and/or an infectious agent (e.g., a virus). In some embodiments, a topical formulation for administration to an eye disclosed herein is administered before or after a physical trauma (e.g., surgery). In some embodiments, the topical formulations described herein are administered for conditions associated with the outer surface of the eye. In other embodiments, the topical formulations are administered to the outer surface of the eye, but subsequently penetrate into the eye to treat a disease associated with the interior of the eye. In other embodiments, the topical formulations are administered in the area of the eye or on the outer surface of the eye in order to treat tissues in contact with or near the eye: non-limiting examples include the tear duct, the eye lid, the eye lash, and the eye socket.

In one aspect, described herein is an ophthalmic formulation comprising a FLAP inhibitor compound in an amount effective for the treatment of an ophthalmic disease, disorder or condition, and at least one suitable pharmaceutically acceptable excipient to provide a solution, suspension, ointment, cream, lotion, niosome, pharmacosome, ointment, or gel.

Provided herein is an ophthalmic formulation comprising a FLAP inhibitor compound in an amount effective for inhibiting leukotriene synthesis, and at least one suitable pharmaceutically acceptable excipient to provide a solution, suspension, ointment, cream, lotion, niosome, pharmacosome, ointment, or gel. In some embodiments, the inhibited leukotriene synthesis results in a reduction in the levels of a cysteinyl leukotriene. In some embodiments, the inhibited leukotriene synthesis results in a reduction in the levels of LTB4.

Provided herein is an ophthalmic formulation comprising a FLAP inhibitor in an amount effective for antagonizing a leukotriene receptor, and suitable pharmaceutically acceptable excipients to provide a solution, suspension, ointment, cream, lotion, niosome, pharmacosome, ointment, or gel.

Provided herein is an ophthalmic formulation comprising a FLAP inhibitor in an amount effective for inhibiting Interleukin-4 (IL-4) synthesis in the eye of an individual.

Provided herein is an ophthalmic formulation comprising a FLAP inhibitor in an amount effective for reducing or inhibiting the synthesis of mucus in the eye of an individual.

In some embodiments, any ophthalmic formulation described herein further comprises a therapeutically-effective amount of a compound selected from antibiotics; anti-fungal agents; steroid anti-inflammatory agents; non-steroidal anti-inflammatory agents; antihistamines; antivirals; alpha agonists; beta blockers; carbonic anhydrase inhibitors; miotics; prostaglandins; anti-angiogenesis agents; loteprednol etabonate, mast cell stabilizers, cyclosporine, and DP2 antagonists.

In some embodiments, any ophthalmic formulation described above further comprises a therapeutically-effective amount of a DP2 receptor antagonist compound.

In one aspect, the ophthalmic disease, disorder or condition is an immune disorder; a proliferation disorder; contact with an allergen and/or an irritant; a fibroblast disorder; an infection (e.g., a viral infection) or combinations thereof.

In another aspect, the ophthalmic disease, disorder or condition is a age-related macular degeneration, allergic conjunctivitis, anterior segment scarring, blepharitis, blepharoconjunctivitis, a bullous disorder, cicatricial pemphigoid, conjunctival melanoma, conjunctivitis, contact lens-associated giant papillary conjunctivitis, diabetic retinopathy, dry eye, episcleritis, glaucoma, gliosis, granuloma annulare, Graves' ophthalmopathy, intraocular melanoma, keratitis, keratoconjunctivitis, pain, pinguecula, post-surgical pain, proliferative vitreoretinopathy, pterygia, scarring, scleritis, Sjögren's syndrome, uveitis, vernal keratoconjunctivitis or combinations thereof.

Provided herein is a method of treating of an ophthalmic disease, disorder or condition comprising administering to an eye of an individual in need thereof an ophthalmic formulation comprising a therapeutically-effective amount of a FLAP inhibitor compound.

Provided herein is a method of inhibiting leukotriene synthesis in the eye of an individual comprising administering to an eye of an individual in need thereof an ophthalmic formulation comprising a FLAP inhibitor compound. In some embodiments, the inhibition of leukotriene synthesis results in a reduction in the level of a cysteinyl leukotriene. In some embodiments, the inhibition of leukotriene synthesis results in a reduction in the level of LTB4.

Provided herein is a method of antagonizing a leukotriene receptor in the eye of an individual in need thereof comprising administering to an eye of an individual an ophthalmic formulation comprising a FLAP inhibitor compound.

Provided herein is a method of inhibiting Interleukin-4 (IL-4) synthesis in the eye of an individual in need thereof, comprising administering to an eye of an individual an ophthalmic formulation comprising a FLAP inhibitor compound.

Provided herein is a method of reducing or inhibiting the synthesis of mucus in the eye of an individual in need thereof, comprising administering to an eye of an individual an ophthalmic formulation comprising a FLAP inhibitor compound.

In one aspect, any method described herein further comprises administering to an eye of an individual in need thereof an ophthalmic formulation comprising a therapeutically-effective amount of a compound selected from antibiotics; anti-fungal agents; steroid anti-inflammatory agents; non-steroidal anti-inflammatory agents; antihistamines; antivirals; alpha agonists; beta blockers; carbonic anhydrase inhibitors; miotics; prostaglandins; anti-angiogenesis agents; loteprednol etabonate, mast cell stabilizers, cyclosporine, and DP2 antagonists, and combinations thereof.

In one aspect, any method described herein further comprises administering to an eye of an individual in need thereof an ophthalmic formulation comprising a therapeutically-effective amount of a DP₂ receptor antagonist compound.

In one aspect, the ophthalmic formulation is in the form of a solution, a suspension, an ointment, a gel, a cream, a liposome, a niosome, a pharmacosome, a nanoparticle, or combinations thereof.

In other aspects, the ophthalmic formulation is administered via implantation, insertion, injection, spraying, washing, or combinations thereof.

In some embodiments, the ophthalmic disease, disorder or condition is an immune disorder; a proliferation disorder; contact with an allergen and/or an irritant; a fibroblast disorder; or combinations thereof.

In some embodiments, the ophthalmic disease, disorder or condition is a age-related macular degeneration, allergic conjunctivitis, anterior segment scarring, blepharitis, blepharoconjunctivitis, a bullous disorder, cicatricial pemphigoid, conjunctival melanoma, conjunctivitis, contact lens-associated giant papillary conjunctivitis, diabetic retinopathy, dry eye, episcleritis, glaucoma, gliosis, granuloma annulare, Graves' ophthalmopathy, intraocular melanoma, keratitis, keratoconjunctivitis, pain, pinguecula, post-surgical pain, proliferative vitreoretinopathy, pterygia, scarring, scleritis, Sjögren's syndrome, uveitis, vernal keratoconjunctivitis or combinations thereof.

In one aspect is the use of a FLAP inhibitor compound in the manufacture of an ophthalmic formulation. In one aspect is the use of a combination of a FLAP inhibitor compound and a DP₂ receptor antagonist compound in the manufacture of an ophthalmic formulation.

In one aspect is the use of a FLAP inhibitor compound in the manufacture of an ophthalmic formulation for the treatment of an ophthalmic disease, disorder or condition. In one aspect is the use of a combination of a FLAP inhibitor compound and a compound selected from antibiotics; anti-fungal agents; steroid anti-inflammatory agents; non-steroidal anti-inflammatory agents; antihistamines; antivirals; alpha agonists; beta blockers; carbonic anhydrase inhibitors; miotics; prostaglandins; anti-angiogenesis agents; loteprednol etabonate, mast cell stabilizers, cyclosporine, and DP2 antagonists; in the manufacture of an ophthalmic formulation for the treatment of an ophthalmic disorder.

### BRIEF DESCRIPTION OF THE FIGURES

FIG. 1 presents illustrative examples of FLAP inhibitor compounds described herein.

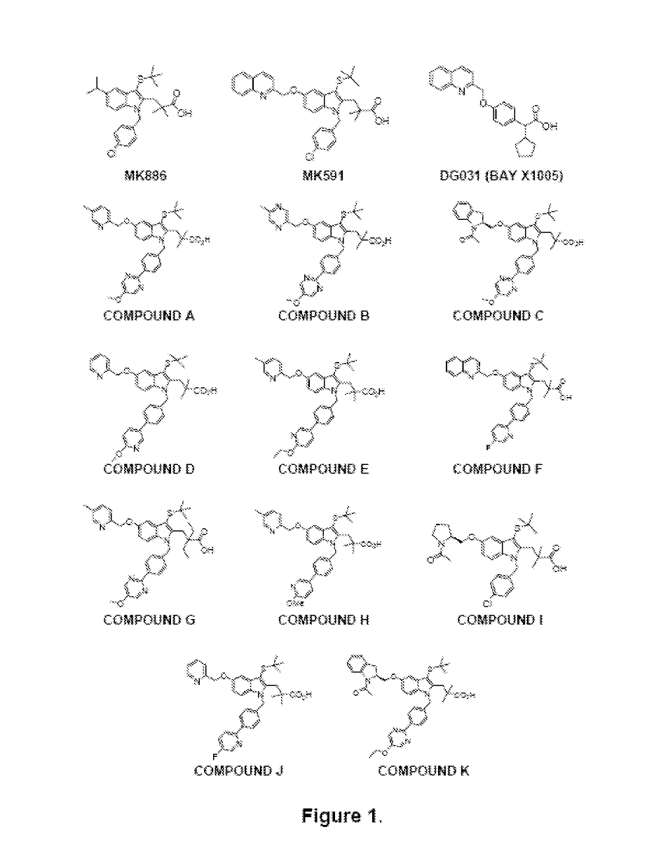

FIG. 2 illustrates the effect of the FLAP inhibitor Compound C on RSV eye pathology.

FIG. 3 illustrates the effect of topical treatment with Compound C on the levels of CysLTs in the eyes of RSV-infected mice.

FIG. 4 illustrates effect of topical treatment with Compound C on the levels of IL-4 in the eyes of RSV-infected mice.

FIG. 5 illustrates RSV plaque forming unit (pfu) in eye and lung.

FIG. 6 illustrates the effect of FLAP inhibition and DP₂ receptor antagonism on the number of total cells, neutrophils and lymhocytes present in bronchoalveolar lavage fluid (BALF).

FIG. 7 illustrates the effect of a combination of a FLAP inhibitor and a DP₂ receptor antagonist on the presence of mucin in BALF.

### DETAILED DESCRIPTION OF THE INVENTION

Leukotrienes are a class of pro-inflammatory lipid mediators derived from arachidonic acid that have been shown to play important roles in a number of biological processes. Arachidonic acid is converted to leukotriene A₄ (LTA₄) in a two-step process mediated by the enzyme 5-lipoxygenase (5-LO). LTA₄ is converted either to LTB₄ via LTA₄ hydrolase or to LTC₄ through conjugation with glutathione mediated by LTC₄ synthase. Amide bond cleavage converts LTC₄ to LTD₄ and then subsequently to LTE₄. The initial oxidation step is a process that requires the intimate involvement of both 5-LO and the membrane bound 5-lipoxygenase-activating protein (FLAP). Inhibition of FLAP results in the inhibition of all leukotriene production. LTB₄ is the ligand for the G protein-coupled receptors (GPCRs) BLT₁ and BLT₂ and both receptors are involved in chemotaxis and cell stimulation in the inflammatory response.

Leukotrienes are lipid mediators of inflammation that are involved in the pathogenesis of ophthalmic diseases, disorders or conditions. Leukotrienes are produced mainly by mast cells, eosinophils, monocytes/macrophages, and neutrophils in response to allergic or inflammatory stimuli. In one aspect, biological tissues in areas that are affected by an ophthalmic disease, disorder or condition have high levels of leukotrienes. The role of FLAP in the leukotriene synthesis pathway is significant because FLAP in concert with 5-lipoxygenase performs the first step in the pathway for the synthesis of leukotrienes. Inhibiting FLAP provides a target for the treatment of leukotriene-dependent or leukotriene mediated ophthalmic diseases, disorders or conditions, including, by way of example, age-related macular degeneration, allergic conjunctivitis, anterior segment scarring, blepharitis, blepharoconjunctivitis, a bullous disorder, cicatricial pemphigoid, conjunctival melanoma, conjunctivitis, contact lens-associated giant papillary conjunctivitis, diabetic retinopathy, dry eye, episcleritis, glaucoma, gliosis, granuloma annulare, Graves' ophthalmopathy, intraocular melanoma, keratitis, keratoconjunctivitis, pain, pinguecula, post-surgical pain, proliferative vitreoretinopathy, pterygia, scarring, scleritis, Sjögren's syndrome, uveitis, vernal keratoconjunctivitis or combinations thereof.

In one aspect, leukotrienes are involved in the pathogenesis of ophthalmic diseases, disorders or conditions. Ophthalmic diseases, disorders or conditions are treated or prevented by ophthalmic administration of a pharmaceutical composition that includes a FLAP inhibitor compound.

Disclosed herein is the use of FLAP inhibitors in the manufacture of medicaments suitable for topical administration to an eye of a mammal for the treatment or prevention of leukotriene-dependent or leukotriene mediated ophthalmic diseases, disorders or conditions.

Described herein are pharmaceutical formulations suitable for topical administration to an eye methods for treating, methods for formulating a topical formulation for administration to an eye, methods for producing, methods for manufacturing, treatment strategies, using a FLAP inhibitor.

Described herein, in certain embodiments, are topical formulations for administration to an eye that include a FLAP inhibitor compound, wherein the formulation is administered to treat an ophthalmic disease, disorder or condition. In one aspect, topical administration of a FLAP inhibitor compound to an eye of a mammal minimizes systemic absorption of the FLAP inhibitor compound. In one aspect, topical administration of a FLAP inhibitor compound to an eye provides for local treatment of an ophthalmic disease, disorder or condition. In one aspect, local treatment of an ophthalmic disease, disorder or condition with a FLAP inhibitor compound reduces possible side effects associated with systemic administration of a FLAP inhibitor compound.

In some embodiments, a topical formulation described herein comprises a FLAP inhibitor compound in combination with an antibiotic; anti-fungal agent; steroid anti-inflammatory agent; non-steroidal anti-inflammatory agent; antihistamine; antiviral; alpha agonist; beta blocker; carbonic anhydrase inhibitor; miotic; prostaglandin; anti-angiogenesis agent; loteprednol etabonate, mast cell stabilizer, cyclosporine, and/or DP2 antagonist to treat or prevent an ophthalmic disease, disorder or condition.

In one aspect, the ophthalmic disease, disorder or condition is a result of the over-production of leukotrienes and/or cytokines. In one aspect, the ophthalmic disease, disorder or condition includes, but is not limited to, ophthalmic immune disorders, ophthalmic proliferative disorders, an ophthalmic disorder resulting from contact with an allergen and/or an irritant, an ophthalmic fibroblast disorder, an infection or combinations thereof.

Ophthalmic immune disorders include, but are not limited to, age-related macular degeneration, allergic conjunctivitis, anterior segment scarring, blepharitis, blepharoconjunctivitis, a bullous disorder, cicatricial pemphigoid, conjunctival melanoma, conjunctivitis, contact lens-associated giant papillary conjunctivitis, diabetic retinopathy, dry eye, episcleritis, glaucoma, gliosis, granuloma annulare, Graves' ophthalmopathy, intraocular melanoma, keratitis, keratoconjunctivitis, pain, pinguecula, post-surgical pain, proliferative vitreoretinopathy, pterygia, scarring, scleritis, Sjögren's syndrome, uveitis, vernal keratoconjunctivitis or combinations thereof.

Ophthalmic proliferative disorders include but are not limited to, intraocular melanoma and conjunctival melanoma. Ophthalmic infections include infections caused by bacteria (e.g., Staphylococcus aureus), viruses (e.g., Respiratory Syntactic Virus (RSV), chlamydia) or the like.

Described herein, in certain embodiments, are topical formulations for administration to an eye that include a FLAP inhibitor compound, wherein the formulation is administered to treat an ophthalmic disease, disorder or condition. In some embodiments, a topical formulation for administration to an eye disclosed herein comprises a therapeutically-effective amount of a FLAP inhibitor. In some embodiments, a topical formulation for administration to an eye disclosed herein is administered before or after contact with an allergen and/or irritant. In some embodiments, a topical formulation for administration to an eye disclosed herein is administered before or after a physical trauma (e.g., surgery). In one aspect, a topical formulation for administration to an eye disclosed herein that includes a FLAP inhibitor compound is administered to an eye and/or a tissue related thereto to treat and prevent scar formation following surgery. It is understood that a topical formulation for administration to an eye disclosed herein is applied to the site of injury. In some embodiments, a topical formulation described herein comprises a FLAP inhibitor compound in combination with an additional therapeutic agent (e.g. NSAID, DP₂ receptor antagonist) and is administered to an eye and/or a tissue related thereto to treat and prevent scar formation following surgery.

In certain instances, leukotrienes are involved in scarring and/or the migration of fibroblasts. In one aspect, inhibiting the activity of FLAP inhibits the activity of and/or migration of fibroblasts, and/or treats scarring. In some embodiments, a topical formulation described herein comprises a FLAP inhibitor compound administered to an eye and/or a tissue related and inhibits the activity of and/or migration of fibroblasts, and/or treats scarring. In some embodiments, a topical formulation described herein comprises a FLAP inhibitor compound in combination with an additional therapeutic agent (e.g. DP₂ receptor antagonist) and is administered to an eye and/or a tissue related and inhibits the activity of and/or migration of fibroblasts, and/or treats scarring.

In one aspect, leukotrienes are involved in the pathogenesis of ophthalmic diseases, disorders or conditions described herein. Inhibition of FLAP will result in a decrease in the production of leukotrienes. A reduction of the amount of leukotrienes results in a decrease of the symptoms associated with such ophthalmic diseases, disorders or conditions. In one aspect, the reduced amounts of leukotrienes are reduced amount of cysteinyl leukotrienes.

In one aspect, the reduced amounts of leukotrienes are reduced amount of LTB4. In some embodiments, a topical formulation described herein comprises a FLAP inhibitor compound in combination with an additional therapeutic agent (e.g. DP₂ receptor antagonist) and is administered to an eye and/or a tissue related and inhibits the production of leukotrienes. In some embodiments, a topical formulation described herein comprises a FLAP inhibitor compound administered to an eye and/or a tissue related and inhibits the production of leukotrienes.

In one aspect, cytokines are involved in the pathogenesis of ophthalmic diseases, disorders or conditions described herein. In some instances, inhibition of FLAP will result in a decrease in the production of at least one cytokine. In one aspect, the cytokine is interleukin 4 (IL-4). A reduction of IL-4 results in a decrease of the symptoms associated with such ophthalmic disorders. In some embodiments, a topical formulation described herein comprises a FLAP inhibitor compound administered to an eye and/or a tissue related and inhibits the production of cytokines. In some embodiments, a topical formulation described herein comprises a FLAP inhibitor compound in combination with an additional therapeutic agent (e.g. DP₂ receptor antagonist) and is administered to an eye and/or a tissue related and inhibits the production of cytokines.

In one aspect, production of lachrymal secretions (e.g., mucus, mucin, pus or the like) is involved in the pathogenesis of ophthalmic diseases, disorders or conditions described herein. Inhibition of FLAP will result in a decrease in the production of lachrymal secretions. A reduction of lachrymal secretions (e.g. mucus production) results in a decrease of the symptoms associated with such ophthalmic diseases, disorders or conditions. In some embodiments, a topical formulation described herein comprises a FLAP inhibitor compound administered to an eye and/or a tissue related and inhibits the production of mucus and/or mucin in the eye of a mammal. In some embodiments, a topical formulation described herein comprises a FLAP inhibitor compound in combination with an additional therapeutic agent (e.g. DP₂ receptor antagonist) and is administered to an eye and/or a tissue related and inhibits the production of mucus and/or mucin in the eye of a mammal.

In one aspect, production of certain glycoslyated proteins known as mucins is involved in the pathogenesis of ophthalmic diseases, disorders or conditions described herein. Inhibition of FLAP will result in a decrease in the production of mucins. A reduction of mucin production results in a decrease of the symptoms associated with such ophthalmic diseases, disorders or conditions. In one aspect, the reduced amounts of mucins are a reduced amount of secreted mucins. In one aspect, the secreted mucins include MUC4 and MUC7 proteins. In another aspect, the reduced amounts of mucins are a reduced amount of gel-forming mucins. In one aspect, the gel-forming mucins include MUC5-AC protein. In yet another aspect, the reduced amounts of mucins are a reduced amount of membrane-associated mucins. In one aspect, the membrane-associated mucins include MUC 1 and MUC16 proteins. In some embodiments, a topical formulation described herein comprises a FLAP inhibitor compound administered to an eye and/or a tissue related and inhibits the protein synthesis of mucin in the eye of a mammal. In other embodiments, a topical formulation described herein comprises a FLAP inhibitor compound administered to an eye and/or a tissue related and inhibits the post-translational modications, for example glycoslyation, of mucin in the eye of a mammal. In some embodiments, a topical formulation described herein comprises a FLAP inhibitor compound administered to an eye and/or a tissue related and increases the protein degradation of mucin in the eye of a mammal.

### FLAP Inhibitors

In one aspect, the FLAP inhibitor compound is selected from FLAP inhibitor compounds disclosed herein or in the art.

In one aspect, the FLAP inhibitor compound is a compound of Formula (I), pharmaceutically acceptable salt, pharmaceutically acceptable solvate, or N-oxide thereof:

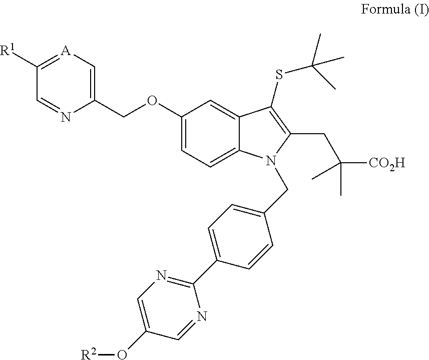

wherein,

### A is CH or N;

R¹ is H, —F, —Cl, —Br, —CN, C₁-C₄alkyl, C₁-C₄-fluoroalkyl, —O—C₁-C₄alkyl, or —O—C₁-C₄-fluoroalkyl;
R² is C₁-C₄alkyl or C₁-C₄-fluoroalkyl.

In one aspect, A is CH. In another aspect, A is N. In one aspect, R¹ is —F, —Cl, —Br, —CN, C₁-C₄alkyl, C₁-C₄-fluoroalkyl, —O—C₁-C₄alkyl, or —O—C₁-C₄-fluoroalkyl. In one aspect, R¹ is —F, —Cl, —Br, —CN, —CH₃, —CH₂CH₃, cyclopropyl, —CF₃, —OCH₃, —OCH₂CH₃, or —OCF₃.

In one aspect, R¹ is H, —F, —Cl, —Br, —CN, —CH₃, —CH₂CH₃, cyclopropyl, —CF₃, —OCH₃, —OCH₂CH₃, or —OCF₃. In another aspect, R¹ is —F, —Cl, —Br, —CN, —CH₃, —CF₃, —OCH₃, or —OCF₃. In another aspect, R¹ is —CH₃.

In one aspect, R² is C₁-C₄alkyl. In another aspect, R² is —CH₃, or —CH₂CH₃. In another aspect, R² is —CH₃.

In one aspect, R¹ is —CH₃ and R² is —CH₃. In one aspect, A is CH, R¹ is —CH₃, and R² is —CH₃.

In one aspect, the FLAP inhibitor compound is 3-[3-tert-Butylsulfanyl-1-[4-(5-methoxy-pyrimidin-2-yl)-benzyl]-5-(5-methyl-pyridin-2-ylmethoxy)-1H-indol-2-yl]-2,2-dimethyl-propionic acid (Compound A); or 3-[3-tert-Butylsulfanyl-1-[4-(5-methoxy-pyrimidin-2-yl)-benzyl]-5-(5-methyl-pyrazin-2-ylmethoxy)-1H-indol-2-yl]-2,2-dimethyl-propionic acid (Compound B); pharmaceutically acceptable salt, pharmaceutically acceptable solvate, or N-oxide thereof. In one aspect, the FLAP inhibitor compound is Compound A; pharmaceutically acceptable salt, pharmaceutically acceptable solvate, or N-oxide thereof.

In another aspect, the FLAP inhibitor compound is a compound of Formula (II), pharmaceutically acceptable salt, pharmaceutically acceptable solvate, or N-oxide thereof:

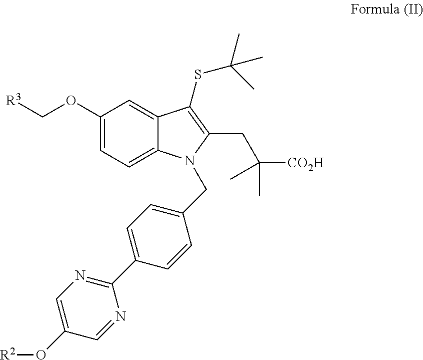

wherein,
R² is C₁-C₄alkyl or C₁-C₄-fluoroalkyl;
R³ is a substituted or unsubstituted monocyclic or bicyclic heterocycolalkyl.

In one aspect, R² is C₁-C₄alkyl. In another aspect, R² is —CH₃, or —CH₂CH₃. In another aspect, R² is —CH₃. In another aspect, R² is —CH₂CH₃.

In one aspect, R³ is a substituted or unsubstituted monocyclic or bicyclic heterocycolalkyl containing at least one N atom in the ring. In one aspect, R³ is a substituted or unsubstituted monocyclic or bicyclic C₃-C₁₀heterocycolalkyl containing at least one N atom in the ring.

In one aspect, R³ is a substituted or unsubstituted monocyclic or bicyclic heterocycloalkyl selected from quinolizinyl, dioxinyl, piperidinyl, morpholinyl, thiazinyl, tetrahydropyridinyl, piperazinyl, oxazinanonyl, dihydropyrrolyl, dihydroimidazolyl, tetrahydrofuranyl, dihydrooxazolyl, oxiranyl, pyrrolidinyl, pyrazolidinyl, imidazolidinonyl, pyrrolidinonyl, dihydrofuranonyl, dioxolanonyl, thiazolidinyl, piperidinonyl, tetrahydronaphyridinyl, tetrahydroquinolinyl, tetrahydrothienyl, indolinyl, and thiazepanyl.

In one aspect, R³ is a substituted or unsubstituted monocyclic or bicyclic heterocycloalkyl selected from piperidinyl, morpholinyl, thiazinyl, tetrahydropyridinyl, piperazinyl, dihydropyrrolyl, dihydroimidazolyl, pyrrolidinyl, pyrazolidinyl, imidazolidinonyl, pyrrolidinonyl, thiazolidinyl, piperidinonyl, tetrahydronaphyridinyl, tetrahydroquinolinyl, and indolinyl.

In one aspect, R³ is a substituted or unsubstituted monocyclic or bicyclic heterocycloalkyl selected from piperidinyl, morpholinyl, piperazinyl, pyrrolidinyl, pyrrolidinonyl, piperidinonyl, tetrahydroquinolinyl, and indolinyl.

In one aspect, R³ is a substituted or unsubstituted monocyclic or bicyclic heterocycloalkyl selected from piperidinyl, morpholinyl, piperazinyl, pyrrolidinyl, tetrahydroquinolinyl, and indolinyl. In one aspect, R³ is a substituted or unsubstituted pyrrolidinyl, and indolinyl. In one aspect, R³ is a substituted or unsubstituted indolinyl.

In further or alternative embodiments, R³ is selected from the group consisting of:

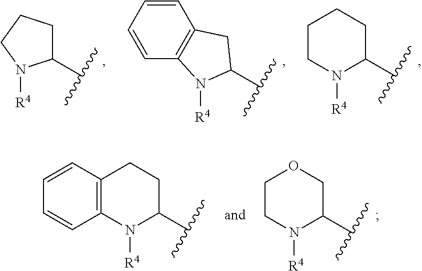

R⁴ is H, —C(═O)R⁵ or —SO₂—C₁-C₄alkyl; R⁵ is C₁-C₄alkyl, C₁-C₄-fluoroalkyl, substituted or unsubstituted phenyl, substituted or unsubstituted heteroaryl, or —O—C₁-C₄alkyl.

In further or alternative embodiments, R³ is selected from the group consisting of:

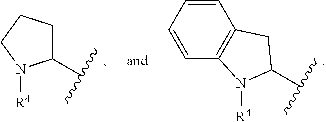

In one aspect, R³ is

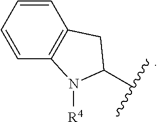

In one aspect, R⁴ is —C(═O)R⁵.

In further or alternative embodiments, R³ is selected from the group consisting of:

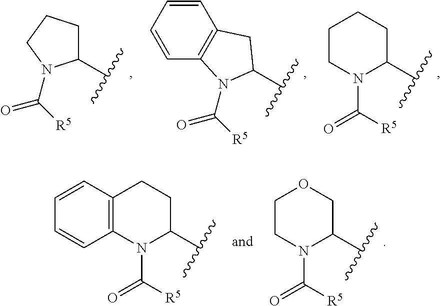

In further or alternative embodiments, R³ is selected from the group consisting of:

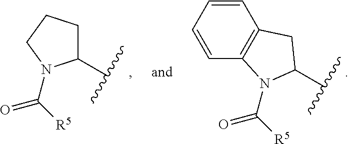

In one aspect, R³ is

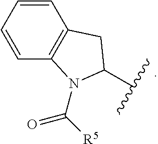

In one aspect, R⁵ is C₁-C₄alkyl, C₁-C₄-fluoroalkyl, substituted or unsubstituted phenyl, or —O—C₁-C₄alkyl. In another aspect, R⁵ is —CH₃, CH₂CH₃, —CH₂CH₂CH₃, —CH₂CH₂CH₂CH₃, —CH(CH₃)₂, —C(CH₃)₃, —CF₃, —OCH₃, —OCH₂CH₃, or —OC(CH₃)₃. In another aspect, R⁵ is —CH₃, —CH₂CH₃, —CH₂CH₂CH₃, —CF₃, —OCH₃, —OCH₂CH₃, or —OC(CH₃)₃. In yet another aspect, R⁵ is —CH₃.

In one aspect, the compound of Formula (II) has the following structure:

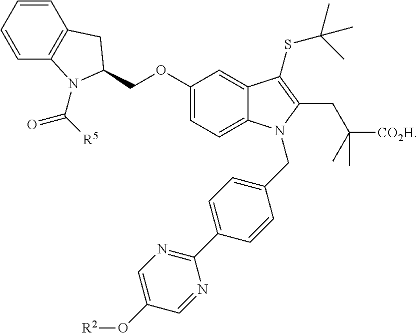

In one aspect, the FLAP inhibitor compound is 3-{5-((S)-1-Acetyl-2,3-dihydro-1H-indol-2-ylmethoxy)-3-tert-butylsulfanyl-1-[4-(5-methoxy-pyrimidin-2-yl)-benzyl]-1H-indol-2-yl}-2,2-dimethyl-propionic acid (Compound C) or 3-{5-((S)-1-Acetyl-2,3-dihydro-1 H-indol-2-ylmethoxy)-3-tert-butylsulfanyl-1-[4-(5-ethoxy-pyrimidin-2-yl)-benzyl]-1H-indol-2-yl}-2,2-dimethyl-propionic acid (Compound K); pharmaceutically acceptable salt, pharmaceutically acceptable solvate, or N-oxide thereof. In one aspect, the FLAP inhibitor compound is Compound C; pharmaceutically acceptable salt, pharmaceutically acceptable solvate, or N-oxide thereof. In one aspect, the FLAP inhibitor compound is Compound K; pharmaceutically acceptable salt, pharmaceutically acceptable solvate, or N-oxide thereof.

In one aspect, the FLAP inhibitor is selected from compounds described in U.S. patent application Ser. No. 11/538,762 (issued as U.S. Pat. No. 7,405,302); U.S. patent application Ser. No. 12/131,828; U.S. patent application Ser. No. 11/553,946 (published as 2007/0105866); U.S. patent application Ser. No. 11/925,841; U.S. patent application Ser. No. 12/089,706; U.S. patent application Ser. No. 12/089,707; U.S. patent application Ser. No. 12/092,570; U.S. patent application Ser. No. 11/744,555 (published as 2007/0219206); U.S. patent application Ser. No. 11/746,010 (published as 2007/0225285); U.S. patent application Ser. No. 11/745,387 (published as 2007/0244128); U.S. patent application Ser. No. 12/257,876; U.S. patent application No. 61/055,887; U.S. patent application No. 61/055,899; International Patent Application no. PCT/US07/86188; WO 07/047,207; WO07/056,021; WO07/056,220; WO07/056,228; International Patent Application no. PCT/US08/62310; International Patent Application no. PCT/US08/062,793; International Patent Application no. PCT/US08/62580; International Patent Application no. PCT/US2008/052960; International Patent Application no. PCT/US08/81190; International Patent Application no. PCT/US08/76225; each of which is herein incorporated by reference in its entirety.

In one aspect, the FLAP inhibitor is selected from: MK886 (also known as 3-[3-tert-butylsulfanyl-1-(4-chloro-benzyl)-5-isopropyl-1H-indol-2-yl]-2,2-dimethyl-propionic acid); MK591 (also known as 3-[3-tert-butylsulfanyl-1-(4-chloro-benzyl)-5-(quinolin-2-ylmethoxy)-1H-indol-2-yl]-2,2-dimethyl-propionic acid); DG031 (also known as BAY X1005; cyclopentyl-[4-(quinolin-2-ylmethoxy)-phenyl]-acetic acid); Compound A (3-[3-tert-Butylsulfanyl-1-[4-(5-methoxy-pyrimidin-2-yl)-benzyl]-5-(5-methyl-pyridin-2-ylmethoxy)-1H-indol-2-yl]-2,2-dimethyl-propionic acid; prepared as outlined in U.S. patent application Ser. No. 11/553,946); Compound B (3-[3-tert-Butylsulfanyl-1-[4-(5-methoxy-pyrimidin-2-yl)-benzyl]-5-(5-methyl-pyrazin-2-ylmethoxy)-1H-indol-2-yl]-2,2-dimethyl-propionic acid; prepared as outlined in U.S. patent application Ser. No. 11/553,946); Compound C (3-{5-((S)-1-Acetyl-2,3-dihydro-1H-indol-2-ylmethoxy)-3-tert-butylsulfanyl-1-[4-(5-methoxy-pyrimidin-2-yl)-benzyl]-1H-indol-2-yl}-2,2-dimethyl-propionic acid; prepared as outlined in WO 07/056,220); Compound D (3-[3-tert-Butylsulfanyl-1-[4-(6-methoxy-pyridin-3-yl)-benzyl]-5-(pyridin-2-ylmethoxy)-1H-indol-2-yl]-2,2-dimethyl-propionic acid; see Compound 2-19 of U.S. patent application Ser. No. 11/553,946); Compound E (3-[3-tert-Butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid; see COMPOUND 2-107 of U.S. patent application Ser. No. 11/553,946); Compound F (3-[3-tert-Butylsulfanyl-1-[4-(5-fluoro-pyridin-2-yl)-benzyl]-5-(quinolin-2-ylmethoxy)-1H-indol-2-yl]-2,2-dimethyl-propionic acid; see Compound 2-197 of U.S. patent application Ser. No. 11/553,946); Compound G (2-[3-tert-Butylsulfanyl-1-[4-(5-methoxy-pyrimidin-2-yl)-benzyl]-5-(5-methyl-pyridin-2-ylmethoxy)-1H-indol-2-ylmethyl]-2-ethyl-butyric acid see Compound 4-38 of U.S. patent application Ser. No. 11/744,555); Compound H (3-[3-tert-Butylsulfanyl-1-[4-(6-methoxy-pyridin-3-yl)-benzyl]-5-(5-methyl-pyridin-2-ylmethoxy)-1H-indol-2-yl]-2,2-dimethyl-propionic acid; see Compound 2-73 of U.S. patent application Ser. No. 11/553,946); Compound I (3-[5-((S)-1-Acetyl-pyrrolidin-2-ylmethoxy)-3-tert-butylsulfanyl-1-(4-chloro-benzyl)-1H-indol-2-yl]-2,2-dimethyl-propionic acid; see Compound 1-2 of WO 07/056,220); Compound J (3-[3-tert-butylsulfanyl-1-[4-(5-fluoro-pyridin-2-yl)-benzyl]-5-(pyridin-2-ylmethoxy)-1H-indol-2-yl]-2,2-dimethyl-propionic acid; see Compound 2-201 of U.S. patent application no. 11/553,946); 3-{5-((S)-1-Acetyl-2,3-dihydro-1H-indol-2-ylmethoxy)-3-tert-butylsulfanyl-1-[4-(5-ethoxy-pyrimidin-2-yl)-benzyl]-1H-indol-2-yl}-2,2-dimethyl-propionic acid (Compound K); pharmaceutically acceptable salt, pharmaceutically acceptable solvate, or
N-oxide thereof.

In another aspect, the FLAP inhibitor is selected from compounds described in U.S. Pat. Nos. 4,929,626; 4,970,215; 5,081,138; 5,095,031; 5,204,344; 5,126,354; 5,221,678; 5,229,516; 5,272,145; 5,283,252; 5,288,743; 5,292,769; 5,304,563; 5,399,699; 5,459,150; 5,512,581; 5,597,833; 5,668,146; 5,668,150; 5,691,351; 5,714,488; 5,783,586; 5,795,900; and 5,843,968, each of which is herein incorporated by reference for the disclosure of such FLAP inhibitors).

### Further Forms of FLAP Inhibitor Compounds

In some embodiments, the therapeutic agent(s) (e.g. FLAP inhibitor compound and/or second therapeutic agent) is present in the pharmaceutical composition as a pharmaceutically acceptable salt. In some embodiments, pharmaceutically acceptable salts are obtained by reacting a FLAP inhibitor compound with acids. In some other embodiments, pharmaceutically acceptable salts are obtained by reacting a FLAP inhibitor compound with a base. In other embodiments, the therapeutic agents are used as free-acid or free-base form in the manufacture of the pharmaceutical compositions described herein. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, FLAP inhibitor compounds described herein are reacted with an organic base, such as, but not limited to, ethanolamine, diethanolamine, triethanolamine, tromethamine, N-methylglucamine, dicyclohexylamine, tris(hydroxymethyl)methylamine. In other cases, FLAP inhibitor compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like. In other cases, FLAP inhibitor compounds described herein form sodium salts and are used as sodium salts.

In some embodiments, the FLAP inhibitor compounds described herein include solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol.

In some embodiments, the FLAP inhibitor compounds described herein possess one or more stereocenters and each center exists independently in either the R or S configuration. The compounds presented herein include all diastereomeric, enantiomeric, and epimeric forms as well as the appropriate mixtures thereof.

In some embodiments, sites on FLAP inhibitor compounds disclosed herein are susceptible to various metabolic reactions Therefore incorporation of appropriate substituents at the places of metabolic reactions will reduce, minimize or eliminate the metabolic pathways. In specific embodiments, the appropriate substituent to decrease or eliminate the susceptibility of the aromatic ring to metabolic reactions is, by way of example only, a halogen, deuterium or an alkyl group.

In some embodiments, FLAP inhibitor compounds described herein are labeled isotopically (e.g. with a radioisotope) or by another other means, including, but not limited to, the use of chromophores or fluorescent moieties, bioluminescent labels, or chemiluminescent labels. In some embodiments, FLAP inhibitor compounds described herein are isotopically-labeled, which are identical to those recited in the various formulae and structures presented herein, but for the fact that one or more atoms are replaced by an atom having an atomic mass or mass number different from the atomic mass or mass number usually found in nature. In some embodiments, one or more hydrogen atoms are replaced with deuterium. In some embodiments, metabolic sites on the compounds described herein are deuterated. In some embodiments, substitution with deuterium affords certain therapeutic advantages resulting from greater metabolic stability, such as, for example, increased in vivo half-life or reduced dosage requirements.

Throughout the specification, groups and substituents thereof can be chosen by one skilled in the field to provide stable moieties and compounds.

### Ophthalmic Diseases, Disorders or Conditions

Described herein, in certain embodiments, are topical formulations for administration to an eye, wherein the formulation is administered to treat an ophthalmic disease, disorder or condition. As used herein, an ophthalmic disease, disorder or condition includes any abnormal state of an eye or a tissue related thereto. In certain instances, an ophthalmic disease, disorder or condition is caused by an immune disorder, (e.g. an autoimmune disorder); a proliferation disorder; contact with an allergen, and/or an irritant; a fibroblast disorder (e.g., scarring after a trauma (e.g., surgery)); or combinations thereof. Ophthalmic diseases, disorders or conditions include, but are not limited to, age-related macular degeneration, allergic conjunctivitis, anterior segment scarring, blepharitis, blepharoconjunctivitis, a bullous disorder, cicatricial pemphigoid, conjunctival melanoma, conjunctivitis, contact lens-associated giant papillary conjunctivitis, diabetic retinopathy, dry eye, episcleritis, glaucoma, gliosis, granuloma annulare, Graves' ophthalmopathy, intraocular melanoma, keratitis, keratoconjunctivitis, pain, pinguecula, post-surgical pain, proliferative vitreoretinopathy, pterygia, scarring, scleritis, Sjögren's syndrome, uveitis, vernal keratoconjunctivitis or combinations thereof.

In some embodiments, a topical formulation for administration to an eye disclosed herein is administered before or after contact with an allergen and/or irritant and/or an infectious agent. In some embodiments, a topical formulation of a FLAP inhibitor for administration to an eye disclosed herein is administered before or after contact with an allergen and/or irritant and/or an infectious agent in combination with antihistamines and/or mast cell stabilizers. In some embodiments, a topical formulation for administration to an eye disclosed herein is administered before or after a physical trauma (e.g., surgery).

In some embodiments, a topical formulation for administration to an eye disclosed herein is administered to treat an ophthalmic disease, disorder or condition, wherein the ophthalmic disease, disorder or condition is conjunctivitis. In certain instances, conjunctivitis results from exposure to an allergen. In certain instances, conjunctivitis results from a bacterial, viral or chlamydial infection. In certain instances, leukotrienes and/or cytokines mediate some or all of the symptoms associated with conjunctivitis. In one aspect, the cytokine is IL-4. In one aspect, symptoms associated with conjunctivitis include, but are not limited to, vessel dilation, edema, hyperemia. In certain instances, inhibiting FLAP activity reduces the concentration of leukotrienes and/or IL-4 associated with conjunctivitis, and, further, treats conjunctivitis. In some embodiments, administration of a FLAP inhibitor compound in combination with an additional therapeutic agent (e.g., antihistamine, mast cell stabilier, DP₂ receptor antagonist) treats conjunctivitis.

In some embodiments, a topical formulation for administration to an eye disclosed herein is administered to treat an ophthalmic disease, disorder or condition, wherein the ophthalmic disease, disorder or condition is keratitis. As used herein, keratitis is a disorder characterized by inflammation of the cornea. In certain instances, leukotrienes and/or cytokines mediate some or all of the symptoms associated with keratitis (e.g., vessel dilation, edema, hyperemia). In certain instances, inhibiting FLAP activity reduces the concentration of leukotrienes and/or cytokines associated with keratitis. In some embodiments, inhibiting FLAP activity treats keratitis. In some embodiments, inhibiting FLAP activity treats vessel dilation, edema, hyperemia, or combinations thereof. In some embodiments, administration of a FLAP inhibitor compound in combination with an additional therapeutic agent (e.g., DP₂ receptor antagonist) treats keratitis.

In some embodiments, a topical formulation for administration to an eye disclosed herein is administered to treat an ophthalmic disease, disorder or condition, wherein the ophthalmic disease, disorder or condition is keratoconjunctivitis (i.e., a combination of conjunctivitis and keratitis (i.e., corneal inflammation)). In certain instances, leukotrienes and/or cytokines mediate some or all of the symptoms associated with keratitis and conjunctivitis (e.g., vessel dilation, edema, hyperemia). In certain instances, inhibiting FLAP activity reduces the concentration of leukotrienes associated with keratitis and conjunctivitis. In some embodiments, inhibiting FLAP activity treats keratoconjunctivitis. In some embodiments, inhibiting FLAP activity treats vessel dilation, edema, hyperemia, or combinations thereof. In some embodiments, administration of a FLAP inhibitor compound in combination with an additional therapeutic agent (e.g. DP₂ receptor antagonist) treats keratitis and conjunctivitis.

In some embodiments, a topical formulation for administration to an eye disclosed herein is administered to treat an ophthalmic disease, disorder or condition, wherein the ophthalmic disease, disorder or condition is blepharitis. As used herein, blepharitis is an ophthalmic disease, disorder or condition characterized by inflammation of the eyelid margins. In certain instances, leukotrienes and/or cytokines mediate some or all of the symptoms associated with blepharitis (e.g., vessel dilation, edema, hyperemia). In certain instances, inhibiting FLAP activity reduces the concentration of leukotrienes associated with blepharitis. In some embodiments, inhibiting FLAP activity treats blepharitis. In some embodiments, inhibiting FLAP activity treats vessel dilation, edema, hyperemia, or combinations thereof. In some embodiments, administration of a FLAP inhibitor compound in combination with an additional therapeutic agent (e.g. DP₂ receptor antagonist) treats blepharitis.

In some embodiments, a topical formulation for administration to an eye disclosed herein is administered to treat an ophthalmic disease, disorder or condition, wherein the disease, disorder or condition is blepharoconjunctivitis (i.e., a combination of conjunctivitis and blepharitis (i.e., inflammation of an eyelid)). In certain instances, leukotrienes mediate some or all of the symptoms associated with blepharitis and conjunctivitis (e.g., vessel dilation, edema, hyperemia). In certain instances, inhibiting FLAP activity reduces the concentration of leukotrienes and/or cytokines associated with blepharitis and conjunctivitis. In some embodiments, inhibiting FLAP activity treats blepharoconjunctivitis. In some embodiments, inhibiting FLAP activity treats vessel dilation, edema, hyperemia, or combinations thereof. In some embodiments, administration of a FLAP inhibitor compound in combination with an additional therapeutic agent (e.g. DP₂ receptor antagonist) treats blepharoconjunctivitis.

In some embodiments, a topical formulation for administration to an eye disclosed herein is administered to treat an ophthalmic disease, disorder or condition, wherein the ophthalmic disease, disorder or condition is scleritis. As used herein, scleritis is a disorder characterized by inflammation of the sclera. In certain instances, leukotrienes mediate some or all of the symptoms associated with scleritis (e.g., vessel dilation, edema, hyperemia). In certain instances, inhibiting FLAP activity reduces the concentration of leukotrienes and/or cytokines associated with scleritis. In some embodiments, inhibiting FLAP activity treats scleritis. In some embodiments, inhibiting FLAP activity treats vessel dilation, edema, hyperemia, or combinations thereof. In some embodiments, administration of a FLAP inhibitor compound in combination with an additional therapeutic agent (e.g., DP₂ receptor antagonist) treats scleritis.

In some embodiments, a topical formulation for administration to an eye disclosed herein is administered to treat an ophthalmic disease, disorder or condition, wherein the ophthalmic disease, disorder or condition is episcleritis. As used herein, episcleritis is an inflammatory disorder of the episclera characterized by hyperaemia, and chemosis. In certain instances, leukotrienes mediate some or all of the symptoms associated with episcleritis (e.g., vessel dilation, edema, hyperemia). In certain instances, inhibiting FLAP activity reduces the concentration of leukotrienes and/or cytokines associated with episcleritis. In some embodiments, inhibiting FLAP activity treats episcleritis. In some embodiments, inhibiting FLAP activity treats vessel dilation, edema, hyperemia, or combinations thereof. In some embodiments, administration of a FLAP inhibitor compound in combination with an additional therapeutic agent (e.g. DP₂ receptor antagonist) treats episcleritis.

In some embodiments, a topical formulation for administration to an eye disclosed herein is administered to treat an ophthalmic disease, disorder or condition, wherein the ophthalmic disease, disorder or condition is uveitis. As used herein, uveitis is an inflammatory disorder of the uvea. In certain instances, leukotrienes mediate some or all of the symptoms associated with uveitis (e.g., vessel dilation, edema, hyperemia). In certain instances, inhibiting FLAP activity reduces the concentration of leukotrienes and/or cytokines associated with uveitis. In some embodiments, inhibiting FLAP activity treats uveitis. In some embodiments, inhibiting FLAP activity treats vessel dilation, edema, hyperemia, or combinations thereof. In some embodiments, administration of a FLAP inhibitor compound in combination with an additional therapeutic agent (e.g. steroid anti-inflammatory agent; non-steroidal anti-inflammatory agent, DP₂ receptor antagonist) treats uveitis.

In some embodiments, a topical formulation for administration to an eye disclosed herein is administered to treat an ophthalmic disease, disorder or condition, wherein the ophthalmic disease, disorder or condition is Sjögren's syndrome. In certain instances, Sjorgren's Syndrome is an autoimmune disorder in which immune cells attack and destroy the exocrine glands that produce tears. In certain instances, leukotrienes mediate some or all of the factors that contribute to the development of Sjorgren's (e.g., the chemotaxis of leukocytes to tear ducts). In certain instances, leukotrienes mediate some or all of the symptoms associated with Graves' (e.g., vessel dilation, edema, hyperemia). In certain instances, inhibiting FLAP activity reduces the concentration of leukotrienes and/or cytokines associated with Sjorgren's Syndrome. In some embodiments, inhibiting FLAP activity treats Sjorgren's Syndrome. In some embodiments, inhibiting FLAP activity inhibits the chemotaxis of leukocytes to tear ducts. In some embodiments, inhibiting FLAP activity treats vessel dilation, edema, hyperemia, or combinations thereof. In some embodiments, administration of a FLAP inhibitor compound in combination with an additional therapeutic agent (e.g., DP₂ receptor antagonist) treats Sjorgen's Syndorome.

In some embodiments, a topical formulation for administration to an eye disclosed herein is administered to treat an ophthalmic disease, disorder or condition, wherein the ophthalmic disease, disorder or condition is Graves' ophthalmopathy (also known as Graves' thyroid-associated or dysthyroid orbitopathy or exophthalmos). Graves' ophthalmopathy is an autoimmune inflammatory disorder affecting the orbit of an eye. In certain instances, an immune system identifies thyroid stimulating hormone receptor (TSH—R) an antigen and attacks it. In certain instances, leukotrienes mediate some or all of the factors that contribute to the development of Graves' ophthalmopathy (e.g., the chemotaxis of leukocytes to the orbit of an eye). In certain instances, leukotrienes mediate some or all of the symptoms associated with Graves' (e.g., vessel dilation, edema, hyperemia). In certain instances, inhibiting FLAP activity reduces the concentration of leukotrienes and/or cytokines associated with Graves' ophthalmopathy. In some embodiments, inhibiting FLAP activity treats Graves' ophthalmopathy. In some embodiments, inhibiting FLAP activity inhibits the chemotaxis of leukocytes to tear ducts. In some embodiments, inhibiting FLAP activity treats vessel dilation, edema, hyperemia, or combinations thereof. In some embodiments, administration of a FLAP inhibitor compound in combination with an additional therapeutic agent (e.g., steroid anti-inflammatory agent, DP₂ receptor antagonist) treats Graves' ophthalmopathy.

In some embodiments, a topical formulation for administration to an eye disclosed herein is administered to treat an ophthalmic disease, disorder or condition, wherein the ophthalmic disease, disorder or condition is a bullous disorder. In certain instances, a bullous disorder is characterized by the formation of blisters (i.e., the accumulation of fluid between cells in a tissue). In certain instances, bullous disorders are autoimmune disorders. In certain instances, leukotrienes mediate the formation of blisters (e.g., induce the exudation of plasma from capillaries to tissues). In certain instances, inhibiting FLAP activity reduces the concentration of leukotrienes and/or cytokines associated with a bullous disorder. In some embodiments, inhibiting FLAP activity treats a bullous disorder. In some embodiments, inhibiting FLAP activity inhibits the exudation of plasma from capillaries to tissues. In some embodiments, administration of a FLAP inhibitor compound in combination with an additional therapeutic agent (e.g., DP₂ receptor antagonist) treats bullous disorders.

In some embodiments, a topical formulation for administration to an eye disclosed herein is administered to treat an ophthalmic disease, disorder or condition, wherein the ophthalmic disease, disorder or condition is scarring. In certain instances, a scar is an area of fibrous tissue that results from the overproduction of collagen. In certain instances, wound healing comprises the migration of fibroblasts to the site of injury. In certain instances, fibroblasts deposit collagen. In certain instances, fibroblasts deposit excess collagen at the wound site, resulting in a scar. In certain instances, leukotrienes modulate the activity of fibroblasts. In certain instances, leukotrienes act as chemotactic agents for fibroblasts. In certain instances, inhibiting the activity of leukotrienes inhibits the activity and/or migration of leukocytes associated with scarring. In certain instances, inhibiting FLAP activity reduces the concentration of leukotrienes and/or cytokines associated with scarring. In certain instances, inhibiting FLAP activity inhibits the activity of and/or migration of fibroblasts associated with scarring. In certain instances, inhibiting FLAP treats scarring. In one aspect, the scarring is anterior segment scarring. In one aspect, the scarring is a result of another ocular disease, disorder or condition. In one aspect, the scarring is a result of another ocular disease, disorder or condition such as, but not limited to, conjuctivitis, keratoconjunctivis. In one aspect, the surface of the eye is scarred. In one aspect, the ophthalmic formulations disclosed herein that include a FLAP inhibitor compound treat or prevent scarring on the surface of the eye of a mammal. In one aspect, the ophthalmic formulations disclosed herein that include a FLAP inhibitor compound treat or prevent post surgical scarring in the eye of a mammal. In one aspect, the combination of non-steroidal anti-inflammatory agent (NSAID) with the ophthalmic formulations disclosed herein that include a FLAP inhibitor compound are used to treat pain and inflammation associated with eye surgery. In some embodiments, administration of a FLAP inhibitor compound in combination with an additional therapeutic agent (e.g., DP₂ receptor antagonist) treats scarring in the eye of a mammal.

In some embodiments, a topical formulation disclosed herein is administered to treat an ophthalmic disease, disorder or condition, wherein the ophthalmic disease, disorder or condition is a melanoma (e.g., intraocular melanoma, and/or conjunctival melanoma). In certain instances, a melanoma is a proliferative disorder of melanocytes. In certain instances, leukotrienes stimulate the growth of melanocytes. Further, in certain instances, inflammation facilitates the growth of a melanoma. In certain instances, leukotrienes mediate inflammation associated with a melanoma. In certain instances, inhibiting FLAP activity reduces the concentration of leukotrienes and/or cytokines associated with a melanoma and slows and/or inhibits the growth of melanocytes associated an intraocular melanoma. In certain instances, inhibiting FLAP activity reduces inflammation associated with a melanoma. In certain instances, inhibiting FLAP treats intraocular melanoma and/or conjunctival melanoma. In some embodiments, administration of a FLAP inhibitor compound in combination with an additional therapeutic agent (e.g., DP₂ receptor antagonist) treats melanoma.

In some embodiments, a topical formulation disclosed herein is administered to treat an ophthalmic disease, disorder or condition, wherein the ophthalmic disease, disorder or condition is an eye infection (e.g., a bacterial, viral or chlamydial infection). In certain instances, an infection causes the production of lachrymal secretions (e.g., mucus, mucin, pus or the like). In certain instances, leukotrienes mediate the production of mucus in an infected eye. In certain instances, inhibiting FLAP activity reduces the concentration of leukotrienes and/or cytokines associated with lachrymal secretions and slows and/or inhibits the production of mucus associated an ocular infection. In certain instances, inhibiting FLAP activity reduces mucus production associated with an eye infection. In certain instances, inhibiting FLAP treats eye infections. In some embodiments, administration of a FLAP inhibitor compound in combination with an additional therapeutic agent (e.g., antibacterial, anti-infective, DP₂ receptor antagonist) treats eye infections.

### Certain Terminology

The terms “individual,” “patient,” or “subject” are used interchangeably. As used herein, they mean any mammal. In one aspect, the mammal is a human.

The terms “treat,” “treating” or “treatment,” and other grammatical equivalents as used herein, include alleviating, abating, inhibiting, reducing, ameliorating, delaying the onset of, arresting the progression of, and/or inducing the regression of a disorder and/or the symptoms of a disorder. The terms also include prophylactic treatment of a disorder. The terms further include achieving any therapeutic benefit. Therapeutic benefit means the eradication or amelioration of the underlying disorder being treated, and/or the eradication or amelioration of one or more of the physiological symptoms associated with the underlying disorder such that an improvement is observed in the individual.

The terms “prevent,” “preventing” or “prevention,” and other grammatical equivalents as used herein include inhibiting (arresting or stopping) the development of a disorder, and/or inhibiting (arresting or stopping) the further progression of a disorder. These terms are intended to include prophylaxis. For prophylactic benefit, a formulation disclosed herein is administered to an individual at risk of developing a particular disorder, or to an individual reporting one or more of the physiological symptoms of a disease, or to an individual at risk of reoccurrence of the disease.

The terms “effective amount” or “therapeutically effective amount” as used herein, refer to an amount of an agent (e.g., FLAP inhibitor compound) being administered which achieve a desired result, e.g., to relieve to some extent one or more symptoms of a disease, disorder or condition being treated. In certain instances, the result is a reduction and/or alleviation of at least one sign, symptom, or cause of a disease, or any other desired alteration of a biological system.

The terms “administer,” “administering,” “administration,” and the like, as used herein, refer to the methods that are used to enable delivery of FLAP inhibitors to the desired site of biological action (e.g., the site of an ophthalmic disorder). These methods include any suitable method for topical administration of a FLAP inhibitor to an eye.

The term “eye” as used herein, includes without limitation, the outer surface and the interior of the eye, the blood vessels in contact with the eye, the orbit and socket of the eye, the epidermal surface and tissues that surround the eye, the eyelid, eyelashes, and fatty deposits surrounding the eye.

### Topical Formulations for Administration to an Eye

In some embodiments, a topical formulation for administration to an eye disclosed herein facilitates the delivery of a FLAP inhibitor compound to the eye or a tissue related thereto for a local effect (i.e., an effect that is limited to the eye or a tissue related thereto). In certain instances, local administration of a FLAP inhibitor compound reduces or eliminates side-effects that are associated with systemic administration of a FLAP inhibitor.

In some embodiments, a FLAP inhibitor for administration to an eye is formulated as a solution, a suspension (e.g., an aqueous suspension), an ointment, a gel, a cream, a liposome, a niosome, a pharmacosome, a nanoparticle, or combinations thereof. In some embodiments, a FLAP inhibitor for topical administration to an eye is administered via implantation, insertion (e.g., via an insoluble insert or a soluble insert), injection, spraying, washing, or combinations thereof.

In some embodiments, a FLAP inhibitor for topical administration to an eye is formulated as a solution, suspension, cream, lotion, ointment, and/or gel. In one embodiment, a FLAP inhibitor is administered as eye drops that can be applied on an eye (or a tissue related thereto) of a mammal, including a human. In one embodiment, a FLAP inhibitor is administered as an eye wash that can be applied on an eye (or a tissue related thereto) of a mammal, including a human.

### Solutions and Suspensions

Disclosed herein, in certain embodiments, is a topical formulation for administration to an eye wherein the topical formulation for administration to an eye is in the form of a solution. Disclosed herein, in certain embodiments, is a topical formulation for administration to an eye wherein the topical formulation for administration to an eye is in the form of a suspension. In certain instances, a solution or suspension rehydrates a tissue and is thus useful for an ophthalmic disease, disorder or condition characterized by loss or reduction of hydration. In certain instances, a solution is an injectable solution for administration (injection) to the eye.

### Creams and Lotions

Disclosed herein, in certain embodiments, is a topical formulation for administration to an eye wherein the topical formulation for administration to an eye is in the form of a cream. In certain instances, creams are semisolid (e.g., soft solid or thick liquid) formulations that include a FLAP inhibitor compound dispersed in an oil-in-water emulsion or a water-in-oil emulsion. Disclosed herein, in certain embodiments, is a topical formulation for administration to an eye wherein the topical formulation for administration to an eye is in the form of a lotion. In certain instances, lotions are fluid emulsions (e.g., oil-in-water emulsions or water-in-oil emulsions). In some embodiments, the hydrophobic component of a lotion and/or cream is derived from an animal (e.g., lanolin, cod liver oil, and ambergris), plant (e.g., safflower oil, castor oil, coconut oil, cottonseed oil, menhaden oil, palm kernel oil, palm oil, peanut oil, soybean oil, rapeseed oil, linseed oil, rice bran oil, pine oil, sesame oil, or sunflower seed oil), or petroleum (e.g., mineral oil, or petroleum jelly).

In certain instances, lotions and creams have a “drying” effect on ophthalmic diseases, disorders or conditions (e.g., some or all fluid exuded from an eye and/or a tissue related thereto is miscible in the ointment) and are thus useful for ophthalmic diseases, disorders or conditions characterized by the exudation of fluids.

### Ointments

Disclosed herein, in certain embodiments, is a topical formulation for administration to an eye wherein the topical formulation for administration to an eye is in the form of an ointment. In certain instances, ointments are semisolid preparations that soften or melt at body temperature (including the temperature of an eye and/or a tissue related thereto). In certain instances, ointments re-hydrate a tissue and are thus useful for ophthalmic diseases, disorders or conditions characterized by loss of moisture.

### Gels

Disclosed herein, in certain embodiments, is a topical formulation for administration to an eye wherein the topical formulation for administration to an eye is in the form of a gel. In certain instances, gels are semisolid (or semi-rigid) systems consisting of dispersions of large organic molecules dispersed in a liquid. In certain instances, gels are water-soluble and are removed using warm water or saline. In certain instances, gels re-hydrate tissues and are thus useful for ophthalmic diseases, disorders or conditions characterized by loss of moisture.

### Ocular-Acceptable Delivery Devices

In some embodiments, a topical formulation for administration to an eye disclosed herein is administered or delivered via a device that can be inserted between an eye and eyelid or in the conjunctival sac, where it releases a FLAP inhibitor. In some embodiments, a topical formulation for administration to an eye disclosed herein is released into the lacrimal fluid that bathes the surface of the cornea, or directly to the cornea itself, with which the solid article is generally in intimate contact. Any suitable device in used with a topical formulation for administration to an eye disclosed herein and methods disclosed herein (e.g., an eyegate applicator).

In some embodiments, a topical formulation for administration to an eye disclosed herein is administered or delivered via an eyedropper. Eyedroppers include any known in the art such as conventional eyedroppers comprising a cylindrical barrel that meters and delivers a dose to an eye when said barrel is deformed under pressure. Other types of eyedroppers include eyedropper types described in U.S. Pat. Nos. 5,514,118; 5,584,823; 5,059,188; 4,834,727; 4,629,456; and 4,515,295, all of which are incorporated by reference for the disclose of eyedropper devices.

### Ocular-Acceptable Injectable Depot Preparations

In some embodiments, a topical formulation for administration to an eye disclosed herein is administered or delivered via an injectable depot preparation. As used herein, a depot preparation is a controlled-release formulation that is implanted in an eye or a tissue related thereto (e.g., the sclera) (for example subcutaneously, intramuscularly, intravitreally, or within the subconjunctiva). The ratio of FLAP inhibitor to controlled-release matrix and the nature of the matrix employed control the rate of drug release.

In some embodiments, a depot preparation is formulated by forming microencapsulated matrices (also known as microencapsule matrices) of a FLAP inhibitor in biodegradable polymers. In some embodiments, a depot preparation is formulated by entrapping a FLAP inhibitor in liposomes or microemulsions.

### Ocular-Acceptable Excipients

In some embodiments, a topical formulation for administration to an eye disclosed herein comprises one or more excipients including carriers, tear substitutes, tonicity enhancers, pH adjusting agents, preservatives, clarifying agents, viscosity enhancers, and solubilizing agents.

In some embodiments, a FLAP inhibitor for administration to an eye formulated as a solution, a suspension, an ointment, a gel, a cream, a liposome, a niosome, a pharmacosome, a nanoparticle, or combinations thereof comprises one or more carriers. Examples of ophthalmically acceptable carriers include, but are not limited to, water, mixtures of water and water-miscible solvents, such as C₁- to C₇-alkanols; vegetable oils or mineral oils comprising from 0.5 to 5% non-toxic water-soluble polymers; natural products, including gelatin, alginates, pectins, tragacanth, karaya gum, xanthan gum, carrageenin, agar and acacia; starch derivatives, including starch acetate and hydroxypropyl starch; and other synthetic products, including polyvinyl alcohol, polyvinylpyrrolidone, polyvinyl methyl ether, polyethylene oxide, polyacrylic acid, such as neutral Carbopol, or mixtures of those polymers.

In some embodiments, a topical formulation for administration to an eye comprises one or more tear substitutes. Tear substitutes promote good wettability on eye tissues, lubrication and spread of the formulation. Tear substitutes also do not provide discomfort to a user. In certain instances, tear substitutes are comprised of one or more carriers disclosed herein. In other instances, tear substitutes are other excipients in addition to a carrier. Exemplary tear substitutes include, but are not limited to monomeric polyols, including, glycerol, propylene glycol, and ethylene glycol; polymeric polyols including polyethylene glycol; cellulose esters including hydroxypropylmethyl cellulose, carboxy methylcellulose sodium and hydroxy propylcellulose; dextrans including dextran 70; water soluble proteins including gelatin; vinyl polymers, including polyvinyl alcohol, polyvinylpyrrolidone, and povidone; and carbomers, including carbomer 934P, carbomer 941, carbomer 940 and carbomer 974P; and any other tear substitutes known in the art. Other commercially available tear substitutes include, but are not limited to cellulose esters such as Bion Tears®, Celluvisc®, Genteal®, OccuCoat®, Refresh®, Teargen II®, Tears Naturale®, Tears Natural II®, Tears Naturale Free®, and TheraTears®; and polyvinyl alcohols such as Akwa Tears®, HypoTears®, Moisture Eyes®, Murine Lubricating®, and Visine Tears®. Paraffin tear substitutes include Lacri-Lube® ointments. Other commercially available ointments that are used as tear substitutes include Lubrifresh PM®, Moisture Eyes PM® and Refresh PM®.

A topical formulation for administration to an eye has an ophthalmically acceptable tonicity. In certain instances, lacrimal fluid has an isotonicity value equivalent to that of a 0.9% sodium chloride solution or a 2.5% solution of glycerol. In certain instances, an isotonicity value from about 0.6% to about 1.8% sodium chloride equivalency is suitable for topical administration to an eye. In certain instances, a topical formulation for administration to an eye disclosed herein has an osmolarity from about 200 to about 600 mOsm/L. In some embodiments, a topical formulation for administration to an eye disclosed herein is hypotonic and thus requires the addition of any agent suitable to attain the proper tonicity range. Ophthalmically acceptable substances that modulate tonicity include ionic and non-ionic tonicity agents. Non-limiting examples of ionic tonicity agents are alkali metal or earth metal halides, such as, for example, CaCl₂, KBr, KCl, LiCl, NaI, NaBr or NaCl, Na₂S₂O₃, NaHSO₄, Na₂SO₄ or boric acid. Another ionic tonicity agent is ammonium sulfate. Non-ionic tonicity enhancing agents are, for example, urea, glycerol, sorbitol, mannitol, propylene glycol, or dextrose.

In some embodiments, a topical formulation for administration to an eye comprises one or more pH adjusting agents. In certain instances, a topical formulation for administration to an eye disclosed herein has a pH in the range of about 4.0 to about 8.0. In certain instances, formulation for administration to an eye has a pH equivalent to tear fluid. pH adjusting agents include, but are not limited to, boric acid, sodium borate, potassium citrate, citric acid, sodium bicarbonate, TRIS, and various mixed phosphate buffers (including combinations of Na₂HPO₄, NaH₂PO₄ and KH₂PO₄) and mixtures thereof.

A topical formulation for administration to an eye has an ophthalmically acceptable clarity. In certain instances, a formulation that lacks suitable clarity interferes with the proper functioning of an eye. Examples of ophthalmically-acceptable clarifying agents include, but are not limited to, polysorbate 20, polysorbate 80, or combinations thereof.

In some embodiments, a topical formulation for administration to an eye comprises an ophthalmically acceptable viscosity enhancer. In certain instances, a viscosity enhancer increases the time a formulation disclosed herein remains in an eye. In certain instances, increasing the time a formulation disclosed herein remains in the eye allows for greater drug absorption and effect. Non-limiting examples of mucoadhesive polymers include carboxymethylcellulose, carbomer (acrylic acid polymer), poly(methylmethacrylate), polyacrylamide, polycarbophil, acrylic acid/butyl acrylate copolymer, sodium alginate, dextrans, hyaluronic acid and its salts, and chondroitin sulfate and its salts.

In some embodiments, a topical formulation for administration to an eye disclosed herein is administered or delivered to the posterior segments of an eye (e.g., to the retina, choroid, vitreous and optic nerve). In some embodiments, a topical formulation for administration to an eye disclosed herein for delivery to the posterior of the eye comprises a solubilizing agent, for example, a glucan sulfate and/or a cyclodextrin. Glucan sulfates which can be used include, but are not limited to, dextran sulfate, cyclodextrin sulfate and β-1,3-glucan sulfate, both natural and derivatives thereof, or any compound which can temporarily bind to and be retained at tissues which contain fibroblast growth factor (FGF), which improves the stability and/or solubility of a drug, and/or which improves penetration and opthalmic absorption of a topical formulation for administration to an eye disclosed herein. Cyclodextrin derivatives that can be used as a solubilizing agent include, but are not limited to, α-cyclodextrin, β-cyclodextrin, γ-cyclodextrin, hydroxyethyl β-cyclodextrin, hydroxypropyl γ-cyclodextrin, hydroxypropyl β-cyclodextrin, sulfated β-cyclodextrin, sulfated α-cyclodextrin, sulfobutyl ether β-cyclodextrin.

In some embodiments, a topical formulation for administration to an eye disclosed herein comprises a preservative. A preservative is added to a formulation to prevent the growth of, or to destroy microorganisms such as fungi or bacteria. Examples of preservatives suitable for topical formulations for administration to an eye include but are not limited to quaternary ammonium salts such as benzalkonium chloride (N-benzyl-N—(C₈-C₁₈ alkyl)-N,N-dimethylammonium chloride), benzoxonium chloride or the like; alkyl-mercury salts of thiosalicylic acid including thiomersal, phenylmercuric nitrate, phenylmercuric acetate and phenylmercuric borate; sodium perborate, sodium chlorite; parabens, including, for example, methylparaben or propylparaben; alcohols, including, for example, chlorobutanol, benzyl alcohol or phenyl ethanol; guanidine derivatives, including, for example, chlorohexidine or polyhexamethylene biguanide, sodium perborate, Germal®II or sorbic acid.

A topical formulation for administration to an eye disclosed herein is formulated in any suitable manner. Any suitable technique, carrier, and/or excipient is contemplated for use with the FLAP inhibitors disclosed herein. For a summary of topical formulations for administration to an eye described herein see Kaur, I. P., Kanwar, M., Drug Dev Industrial Pharmacy, 2002, 28, 473-493; Lang, J. C., Adv Drug Delivery Rev., 1995, 16, 39-43; Remington: The Science and Practice of Pharmacy, Nineteenth Ed. (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), which are herein incorporated by reference for such disclosures.

### Dosing

Disclosed herein, in certain embodiments, is a topical formulation for administration to an eye, wherein the topical formulation for administration to an eye is administered for prophylactic and/or therapeutic treatments. In certain instances, amounts effective for this use will depend on the severity and course of the disease, disorder or condition, previous therapy, the individual's health status and response to the drugs, and the judgment of the treating physician. In some embodiments, the dose is about 0.001% by weight to about 10% by weight.

The compounds described herein are optionally administered to any portion of the eye, generally referred to as “ophthalmic administration.” Ophthalmic administration to the eye encompasses, but is not limited to, intraocular injection, subretinal injection, intravitreal injection, periocular administration, subconjuctival injections, retrobulbar injections, intracameral injections (including into the anterior or vitreous chamber), sub-Tenon's injections or implants, ophthalmic solutions, ophthalmic suspensions, ophthalmic ointments, ocular implants and ocular inserts, intraocular solutions, use of iontophoresis, incorporation in surgical irrigating solutions, and packs (by way of example only, a saturated cotton pledget inserted in the formix).

Administration of a topical formulation for administration to an eye disclosed herein generally results in direct contact of a FLAP inhibitor with the cornea, through which at least a portion of a FLAP inhibitor passes. In certain instances, a topical formulation for administration to an eye disclosed herein has an effective residence time in an eye of about 2 to about 24 hours, more typically about 4 to about 24 hours and most typically about 6 to about 24 hours.

Useful topical formulations for administration to an eye can be an aqueous solution, suspension or solution/suspension, which can be presented in the form of eye drops. A desired dosage can be administered via a set number of drops into an eye. For example, for a drop volume of 25 μl, administration of 1-6 drops will deliver 25-150 μl of a topical formulation for administration to an eye disclosed herein. Aqueous formulations typically contain from about 0.01% to about 50%, more typically about 0.1% to about 20%, still more typically about 0.2% to about 10%, and most typically about 0.5% to about 5%, weight/volume of a FLAP inhibitor.

In some embodiments, topical formulations for administration to an eye are administered by placing one or more doses of a solution, suspension, gel, ointment, cream or lotion on a contact lens, and inserting the lens for a set period time, for example, 15 minutes to 4 hours, repeatable 1× to 4× per day. Contact lens dosing allows for advantages such as preferential absorption within the cornea, maximizing dose utilization and minimizing mild redness that may otherwise occur as well as the remote risk of systemic absorption. Useful contact lens include any known in the art and include, but are not limited to, hard hydrophobic lens including polymethyl methacrylate (PMMA) lens; soft, hydrophilic lens including hydroxyethyl methylmethacrylate lens; flexible hydrophobic lens including silicone vinylpyrollidone; and rigid hydrophilic lens including cellulose acetate butyrate lens.

In some embodiments, where an ophthalmic disorders does not improve, a topical formulation for administration to an eye disclosed herein is administered chronically (i.e., for an extended period of time, including throughout the duration of the individual's life). In some embodiments, where an ophthalmic disorder does improve, a topical formulation for administration to an eye disclosed herein is given continuously; alternatively, the dose of FLAP inhibitor being administered is temporarily reduced or temporarily suspended for a certain length of time (i.e., a “drug holiday”). In some embodiments, a drug holiday lasts between 2 days and 1 year, including all integers in between. In some embodiments, the dose reduction during a drug holiday is from about 10% to about 100%, including all integers in between.

In some embodiments, where an ophthalmic disorder does improve, a topical formulation for administration to an eye disclosed herein is administered as a maintenance dose. In some embodiments, where an ophthalmic disorder does improve, a topical formulation for administration to an eye disclosed herein is administered with reduced frequency or at a reduced dose.

In one embodiment, a topical formulation for administration to an eye disclosed herein is formulated for immediate release of a FLAP inhibitor. In some embodiments, a FLAP inhibitor is released immediately, or within 1 minute, or within 5 minutes, or within 10 minutes, or within 15 minutes, or within 30 minutes, or within 60 minutes or within 90 minutes.

In one embodiment, a topical formulation for administration to an eye disclosed herein is formulated for delayed (or controlled) release of a FLAP inhibitor. In some embodiments, a FLAP inhibitor compound is released over a time period exceeding 15 minutes, or 30 minutes, or 1 hour, or 4 hours, or 6 hours, or 12 hours, or 18 hours, or 1 day, or 2 days, or 3 days, or 4 days, or 5 days, or 6 days, or 7 days, or 10 days, or 12 days, or 14 days, or 18 days, or 21 days, or 25 days, or 30 days, or 45 days, or 2 months or 3 months or 4 months or 5 months or 6 months or 9 months or 1 year.

In some embodiments, a topical formulation for administration to an eye disclosed herein is formulated for immediate and delayed (or controlled) release of a FLAP inhibitor.

### Combination Therapy

In one aspect, pharmaceutical compositions and methods disclosed herein include an additional therapeutic agent. In one aspect, the additional therapeutic agent is a therapeutic agent other than a FLAP inhibitor compound.

In one aspect, the ophthalmic formulations disclosed herein that include a FLAP inhibitor compound are co-administered with (either separately or in the same formulation) a therapeutic agent selected from: antibiotics (e.g., polymyxin B sulfate/bacitracin zinc, polymyxin B/neomycin/gramicidin, polymyxin B/trimethoprim, polymyxin B/bacitracin, fluoroquinolones (e.g., ciprofloxacin, moxifloxacin, ofloxacin, gatifloxacin, levofloxacin), aminoglycosides (e.g. tobramycin, azithromycin, gentamicin, erythromycin, bacitracin); anti-Fungal Agents (e.g., amphotericin B, intraconazole, fluconazole, voriconazole); steroid anti-inflammatory agents (e.g., fluorometholone acetate, prednisolone acetate, loteprednol etabonate, prednisolone sodium phosphate, prednisolone sodium, rimexolone, fluorometholone acetate); non-steroidal anti-inflammatory agents (e.g., nepafenac, ketorolac tromethamine, bromfenac, diclofenac sodium, ketorolac tromethamine, ketotifen fumarate); antihistamines (e.g., emedastine difumarate, olopatadine hydrochloride, epinastine HCl, Azelastine Hydrochloride, ketotifen fumarate); antivirals (e.g., acyclovir, vidarabine, trifluridine); alpha agonists (e.g., apraclonidine, brimonidine, bimatoprost); beta blockers (e.g., betaxolol hydrochloride, levobunolol hydrochloride, carteolol hydrochloride, metipranolol, timolol maleate, timolol hemihydrate); carbonic anhydrase inhibitors (e.g., brinzolamide, dorzolamide, acetazolamide); miotics (e.g., acetylcholine chloride, echothiophate); prostaglandins (e.g., travoprost, bimatoprost, latanoprost); anti-angiogenesis agents (e.g., pegaptanib sodium, ranibizumab, verteporfin); loteprednol etabonate, mast cell stabilizers (e.g., lodoxamide tromethamine, nedocromil sodium, cromolyn sodium, pemirolast potassium), cyclosporine, and DP2 antagonists.

In some embodiments, the ophthalmic pharmaceutical compositions disclosed herein comprising a FLAP inhibitor compound are co-administered with (either separately or in the same formulation) an antibiotic. Antibiotics include, but are not limited to polymyxin B sulfate/bacitracin zinc, polymyxin B/neomycin/gramicidin, polymyxin B/trimethoprim, polymyxin B/bacitracin, fluoroquinolones (e.g., ciprofloxacin, moxifloxacin, ofloxacin, gatifloxacin, levofloxacin), aminoglycosides (e.g. tobramycin, azithromycin, gentamicin, erythromycin, bacitracin).

In some embodiments, the ophthalmic pharmaceutical compositions disclosed herein comprising a FLAP inhibitor compound are co-administered with (either separately or in the same formulation) an anti-fungal agent. Anti-fungal agents include, but are not limited to amphotericin B, intraconazole, fluconazole, and voriconazole.

In some embodiments, the ophthalmic pharmaceutical compositions disclosed herein comprising a FLAP inhibitor compound are co-administered with (either separately or in the same formulation) a steroid anti-inflammatory agent. Steroid anti-inflammatory agents include but are not limited to, betamethasone, prednisone, alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, halcinonide, halometasone, hydrocortisone/cortisol, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone/prednisolone, rimexolone, tixocortol, triamcinolone, and ulobetasol.

In some embodiments, the ophthalmic pharmaceutical compositions disclosed herein comprising a FLAP inhibitor compound are co-administered with (either separately or in the same formulation) a non-steroidal anti-inflammatory agent (NSAID). NSAIDs include, but are not limited to, nepafenac, ketorolac, bromfenac, diclofenac, ketorolac, ketotifen.

In some embodiments, the ophthalmic pharmaceutical compositions disclosed herein comprising a FLAP inhibitor compound are co-administered with (either separately or in the same formulation) an antihistamine. In some embodiments, antihistamines include, but are not limited to, amelexanox, astemizole, azatadine, azelastine, acrivastine, brompheniramine, cetirizine, levocetirizine, efletirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, olopatadine, picumast, pyrilamine, promethazine, terfenadine, tripelennamine, temelastine, trimeprazine, and triprolidine. In some embodiments, antihistamines include, but are not limited to, emedastine, olopatadine, epinastine, azelastine, ketotifen.

In some embodiments, the ophthalmic pharmaceutical compositions disclosed herein comprising a FLAP inhibitor compound are co-administered with (either separately or in the same formulation) an antiviral agent. Antiviral agents include, but are not limited to, acyclovir, vidarabine, trifluridine.

In some embodiments, the ophthalmic pharmaceutical compositions disclosed herein comprising a FLAP inhibitor compound are co-administered with (either separately or in the same formulation) and alpha agonist. Alpha agonists include, but are not limited to, apraclonidine, brimonidine, bimatoprost.

In some embodiments, the ophthalmic pharmaceutical compositions disclosed herein comprising a FLAP inhibitor compound are co-administered with (either separately or in the same formulation) a beta blocker. Beta blockers include, but are not limited to, betaxolol, levobunolol, carteolol, metipranolol, timolol.

In some embodiments, the ophthalmic pharmaceutical compositions disclosed herein comprising a FLAP inhibitor compound are co-administered with (either separately or in the same formulation) a carbonic anhydrase inhibitor. Carbonic anhydrase inhibitors include, but are not limited to, brinzolamide, dorzolamide, acetazolamide.

In some embodiments, the ophthalmic pharmaceutical compositions disclosed herein comprising a FLAP inhibitor compound are co-administered with (either separately or in the same formulation) a miotic. Miotics include, but are not limited to, acetylcholine chloride, echothiophate.

In some embodiments, the ophthalmic pharmaceutical compositions disclosed herein comprising a FLAP inhibitor compound are co-administered with (either separately or in the same formulation) a prostaglandin. Prostaglandins include, but are not limited to, travoprost, bimatoprost, latanoprost.

In some embodiments, the ophthalmic pharmaceutical compositions disclosed herein comprising a FLAP inhibitor compound are co-administered with (either separately or in the same formulation) an anti-angiogenesis agent. Anti-angiogenesis agents include, but are not limited to, pegaptanib sodium, ranibizumab, verteporfin.

In some embodiments, the ophthalmic pharmaceutical compositions disclosed herein comprising a FLAP inhibitor compound are co-administered with (either separately or in the same formulation) loteprednol etabonate.

In some embodiments, the ophthalmic pharmaceutical compositions disclosed herein comprising a FLAP inhibitor compound are co-administered with (either separately or in the same formulation) a mast cell stabilizer. Mast cell stabilizers include, but are not limited to, lodoxamide tromethamine, nedocromil sodium, cromolyn sodium, pemirolast potassium.

In some embodiments, the ophthalmic pharmaceutical compositions disclosed herein comprising a FLAP inhibitor compound are co-administered with (either separately or in the same formulation) cyclosporine.

In some embodiments, the ophthalmic pharmaceutical compositions disclosed herein comprising a FLAP inhibitor compound are co-administered with (either separately or in the same formulation) a DP₂ receptor antagonist. In one aspect, the additional therapeutic agent is a small molecule DP₂ receptor antagonist compound. In some embodiments, a DP₂ receptor antagonist is selected from compounds disclosed in International patent application no. PCT/US09/35174 (entitled Antagonists of Prostaglandin D₂ receptors); International patent application no. PCT/US08/82056 (entitled Antagonists of PGD₂ receptors); International patent application no. PCT/US08/82082 (entitled Antagonists of PGD₂ receptors); International patent application no. PCT/US0932495 (entitled N,N-disubstituted aminoalkylbiphenyl antagonists of prostaglandin D₂ receptors); International patent application no. PCT/US09/32499 (entitled “N,N-disubstituted aminoalkylbiphenyl antagonists of prostaglandin D₂ receptors”); International patent application no. PCT/US09/33961 (entitled “Cyclic diaryl ether compounds as antagonists of prostaglandin D₂ receptors”); International patent application no. PCT/US09/38291 (entitled “Aminoalkylphenyl antagonists of prostaglandin D₂ receptors”); International patent application no. PCT/US09/49621 (entitled “Antagonists of prostaglandin D₂ receptors”); International patent application no. PCT/US09/49631 (entitled “Antagonists of prostaglandin D₂ receptors”); International patent application no. PCT/US09/58655 (entitled “Heteroaryl antagonists of prostaglandin D₂ receptors”); International patent application no. PCT/US09/58663 (entitled “Heteroaryl antagonists of prostaglandin D₂ receptors”); International patent application no. PCT/US09/44219 (entitled “Tricyclic antagonists of prostaglandin D₂ receptors”); International patent application no. PCT/US09/48327 (entitled “Cycloaklane[B]indole antagonists of prostaglandin D₂ receptors”); International patent application no. PCT/US09/59256 (entitled “Heteroaryl antagonists of prostaglandin D₂ receptors”); International patent application no. PCT/US09/59891 (entitled “Heteroalkyl biphenyl antagonists of prostaglandin D₂ receptors”); International patent application no. PCT/US09/64630 (entitled “Heterocyclic antagonists of prostaglandin D₂ receptors”); International patent application no. PCT/US09/63439 (entitled “Cycloaklane[B]azaindole antagonists of prostaglandin D2 receptors”); International patent application no. PCT/US09/63438 (entitled “Cycloaklane[B]azaindole antagonists of prostaglandin D2 receptors”); U.S. provisional application No. 61/147,437 (entitled “Indolozine compounds as prostaglandin D₂ receptor antagonists”); or pharmaceutically acceptable salts or N-oxides thereof.

In some embodiments, a DP₂ receptor antagonist is selected from AMG 009, AMG 853, Compound 14 of WO 09/085,177, AZD1981, ODC9101 (OC459), OC499, OC1768, OC2125, OC2184, QAV680, MLN6095, ACT-129968, ADC3680, SAR398171, S555739, AP768, [2′-(3-Benzyl-1-ethyl-ureidomethyl)-6-methoxy-4′-trifluoromethyl-biphenyl-3-yl]-acetic acid, {3-[2-tert-Butylsulfanylmethyl-4-(2,2-dimethyl-propionylamino)-phenoxy]-4-methoxy-phenyl}-acetic acid, TM30642, TM30643, TM30089, TM27632, and TM3170, {2′-[(N-cyclopropanecarbonyl-N-ethyl-amino)-methyl]-6-methoxy-4′-trifluoromethyl-biphenyl-3-yl}-acetic acid, [2′-[(N-cyclopropanecarbonyl-N-ethyl-amino)-methyl]-4′-(6-ethoxy-pyridin-3-yl)-6-methoxy-biphenyl-3-yl]-acetic acid, (5-{2-[(N-benzyloxycarbonyl-N-ethyl-amino)-methyl]-4-trifluoromethyl-phenyl}-pyridin-3-yl)-acetic acid, and {8-[(4-fluoro-benzenesulfonyl)-methyl-amino]-6,7,8,9-tetrahydro-pyrido[3,2-b]indol-5-yl}-acetic acid.

In some instances, the ratio of the amount of a FLAP inhibitor compound to a DP₂ receptor antagonist compound in any ophthalmic formulation or method described herein is from about 10:1 to about 1:10. In some instances, the ratio of the amount of a FLAP inhibitor compound to a DP₂ receptor antagonist in any ophthalmic formulation or method described herein is about 10:1, about 8:1, about 6:1, about 5:1, about 4:1, about 2:1, about 1:1, about 1:2, about 1:4, about 1:5, about 1:6, about 1:8, or about 1:10.

In some embodiments, the FLAP inhibitor and the additional therapeutic agent are in the same pharmaceutical composition. In some embodiments, the FLAP inhibitor and the additional therapeutic agent are in separate pharmaceutical compositions. In some embodiments, the FLAP inhibitor and the additional therapeutic agent are administered at the same time. In some embodiments, the FLAP inhibitor and the additional therapeutic agent are administered at different times.

### EXAMPLES

### Example 1

### Formulation of a FLAP Inhibitor

In one aspect, aqueous ophthalmic solutions that include a FLAP inhibitor compounds were prepared by dissolving a FLAP inhibitor compound in 10% beta-hydroxypropyl cyclodextrin (BHPC) in water. Using this formulation, the following compounds were soluble at 1 mg/mL: Compound A (Na salt), Compound B (Na salt), Compound C (Na salt), Compound I (Na salt), Compound J (Na salt), Compound G (Na salt). Compound F (Na salt) formed a cloudy solution at 1 mg/mL.

### Example 2

### Solution of Compound A

A solution of Compound A suitable for administration to the eye is formulated with the following components:

| | | |
|---|---|---|
| Compound A | 2.0 | g |
| Polyvinyl alcohol | 1.5 | mL |
| Povidone | 0.5 | g |
| Chlorobutanol | 0.5 | mL |
| Polysorbate 20 | 1 | mL |
| Sodium chloride (0.9%) | Qs to 100 | mL |

### Example 3

### Solution of Compound B

A solution of Compound B suitable for administration to the eye is formulated with the following components:

| | | |
|---|---|---|
| Compound B | 2.0 | g |
| Benzalkonium chloride | 0.13 | g |
| Polysorbate 80 | 1 | mL |
| Sodium chloride (0.9%) | Qs to 100 | mL |

### Example 4

### Solution of Compound C

A solution of Compound C suitable for administration to the eye is formulated with the following components:

| | | |
|---|---|---|
| Compound C | 1.5 | g |
| Hydroxyethylcellulose | 0.8 | g |
| Benzalkonium chloride | 0.13 | g |
| Polysorbate 20 | 1 | mL |
| Sodium chloride (0.9%) | Qs to 100 | mL |

### Example 5

### Clinical Trial Evaluating Effect of Topical Administration of a FLAP Inhibitor Compound to the Eye in Reducing Signs and Symptoms of Allergic Conjunctivitis

A single-center, double-blind, randomized, two way cross-over, placebo-controlled study to evaluate the safety and efficacy of topical administration of a FLAP inhibitor compound to the eye of individuals with allergic conjunctivitis (AC) following conjunctival allergen challenge (CAC). Each subject will receive the active treatment (e.g., a topical formulation of a FLAP inhibitor compound administered to the eye) or placebo.

Ten subjects aged 18-65 years of either gender are to participate in the study. All subjects should have a medical history of ocular allergies (allergic conjunctivitis or rhinoconjunctivitis). Their allergic status should have been confirmed by (1) positive skin prick test to a seasonal or perennial allergen, (2) a positive ocular allergen challenge to the same allergen.

No subject should have glaucoma, anterior or posterior uveitis, clinically significant blepharitis, follicular conjunctivitis, iritis or dry eye; diabetic retinopathy, or progressive retinal disease; presence of an active ocular infection; a positive history of an ocular herpetic infection. Also excluded are subjects that have had previous treatment (within 2 weeks prior to randomization) with any systemically administered or ophthalmically administered corticosteroids; or any systemically administered or ophthalmically administered mast cell stabilizers; subjects who had an upper respiratory tract infection 4 weeks before randomization, or subjects who have undergone ocular surgery within 6 months or had a history of retinal detachment. Female subjects of child bearing potential must have a documented negative urine pregnancy test and must be practicing a medically proven form of contraception during the course of the study period. Written informed consent is obtained from each subject.

### Conjunctival Allergen Challenge (CAC) Protocol

Demographic, medical and medication histories are obtained from patients. Patients that are eligible will have CAC performed bilaterally with an allergen to which the patient had previously reacted positively following a skin test. Allergen is instilled at increasing concentrations at 10-minute intervals until a positive reaction is observed. Patients and physician will then assess signs and symptoms of ocular inflammation: ocular itching, tearing, and redness/burning sensation using a 5 point scale (0=none to 4=incapacitating) for each symptom at 3, 7 and 20 minutes and 12 hours post CAC. The patient symptom score is the sum of individual scores as rated by the patient. The concentration of allergen inducing a 30% change in the patient symptom score (PD 30), as measured by the AUC symptom score 0 to 20 minutes, will be established. Each patient will then receive the allergen challenge with PD 30 concentration at day 1 of the first period of the cross over and again at day 1 of the second period of the cross over. Each patient will receive the treatment (active or placebo) immediately after allergen challenge. The two periods of the cross over will be separated by a seven-day wash out period.

The primary endpoint of the study is the variation of patient symptom score (AUC symptom score 0 to 20 minutes) treated versus placebo. Secondary endpoints include the individual ocular signs and symptoms as assessed by the patient and the physician, the overall assessment of the patient, the overall assessment of the physician, safety and tolerability, nasal symptoms, and biomarkers in the lachrymal secretions.

### Example 6

### Rat Model of Allergic Conjunctivitis

A rat model of allergic conjunctivitis is used to test the effect of topical administration of a FLAP inhibitor compound to the eye on the development of allergic conjunctivitis. Male wistar rats (250-350 g) are sensitized by injection with 0.6 mL saline containing ovalbumin (OVA, 1 mg), alum (2 mg) and 10¹⁰ killed B. pertussis cells into all four footpads on day 1. Five days later they are boosted by subcutaneous injection with 1 ml of saline containing OVA (0.5 mg) in 10 sites on the back. Local sensitization is performed daily from days 14 to day 42 by instilling OVA in saline (10 mg/ml, 5 μl) into the bilateral eyes using a micropipette. Rats are treated with systemic or ocular FLAP inhibitor on days 14 to 42 (as appropriate). The frequency of eye scratching behavior is counted for 20 min post OVA on selected days. Twenty-four hours following OVA challenge on days 14, 21, 28, 35 and 42 rats are anesthetized and the conjunctiva removed and fixed with 10% neutral buffered formalin. 4-μm thick frontal sections are stained and eosinophils counted. The inhibition of eye scratching behavior and conjunctiva eosinophils following topical treatment with a FLAP inhibitor compound to the eye(s) is recorded and plotted using Graphpad Prizm.

### Example 7

### Mouse Model of RSV-Infected Eye

A mouse model of respiratory syncytial virus (RSV) eye infection is used to test the effects of ocular application of a FLAP inhibitor compound on RSV-induced ocular immunopathology (Bitko V, et al., J. Virol. 2007; 81(2):783-90; Bitko V, et al., Nat. Med. 2005; 11(1):50-5). Each mouse eye is topically treated with 60 ng of FLAP inhibitor compound or vehicle (sterile saline) in a volume of 2 μl. The FLAP inhibitor compound is instilled 40 min after virus inoculum and then once daily over the entire duration of the protocol (14 days).

Instillation of virus and inhibitor in the eye. Female BALB/c mice, 6 to 8 weeks old, were purchased from Charles River Laboratories. RSV (Long strain, serotype A) was grown on HEp-2 cells and purified on sucrose layers to a concentration of 10¹¹ PFU (Bitko et al., Nat. Med. 11:50-55, 2005). Dilutions were done in phosphate-buffered saline (PBS) immediately before use to a final concentration of 10⁴ PFU/2 μL as needed. A similarly diluted sucrose solution was used in sham-infected control mice. Mice were anesthetized by intraperitoneal injection of pentobarbital (50 mg/kg), and virus in 2 μL PBS was dropped into the corneal surface and massaged in with closed eyelids. The day of the inoculation was considered day 0. The FLAP inhibitor Compound C was diluted in PBS to 60 ng/2 μL just prior to application of 2 μL 40 minutes after the RSV inoculation on day 0 and then each day.

Duration of the experiment: Eyes will are visually examined daily, and harvested on each of the following days post-RSV inoculation: Day 2, 4, 6, 8, 9, 10, 12, 14=a total of 8 time points. Three animals are used for each data point i.e. 24 mice treated with a FLAP inhibitor compound C and 24 mice treated with vehicle control (for a total of 48 mice used in the study).

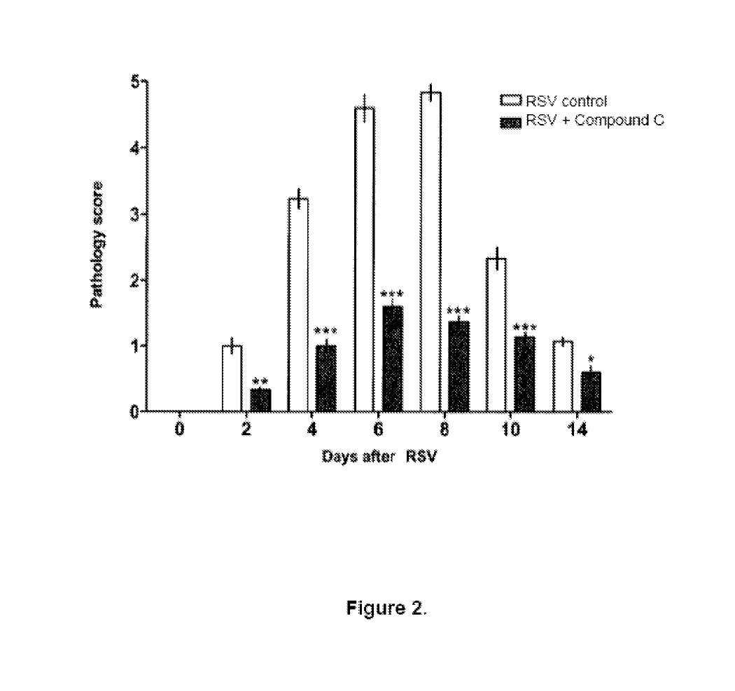

Ocular pathology. Ocular disease was evaluated with a slit lamp biomicroscope as described previously (Bitko V, et al., J. Virol. 2007; 81(2):783-90) 15; Girgis et al., Invest. Ophthal. Vis. Sci. 44:1591-1597, 2003). Pathology was scored on a scale of 0 to 5 as follows: 0=clear eye; 1=slight redness in the corners; 2=moderate redness and injection; 3=conjunctival and corneal injection with ciliary flush; 4=extensive injection, generally associated with some mucus; 5=most extensive injection, associated with mucus. Eyes were examined in a coded fashion with the reader unaware of the treatment given. FIG. 2 illustrates the effect of the FLAP inhibitor Compound C on RSV eye pathology. Ocular application of the FLAP inhibitor Compound C reduces the pathology score across all observation days, i.e, days 2-14 after RSV challenge. The RSV treated eyes showed gummy residue and mucin build up starting at day 4 and heavy mucus build up by day 8. Infected eyes treated with the FLAP inhibitor Compound C were clear of gummy residue and mucin build up on day 8.

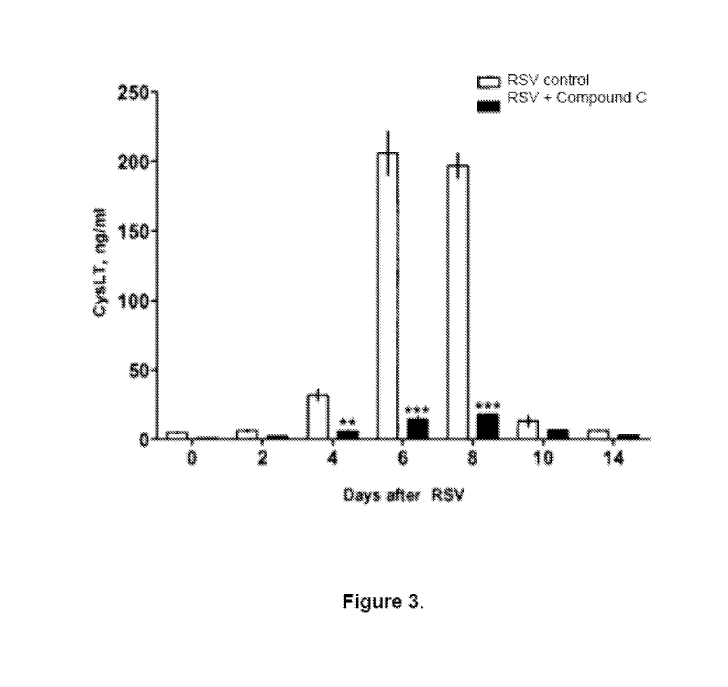

CysLT measurements Eye tissues were homogenized in 1-3 ml lysis buffer (0.5% Triton-X-100, 15 mM Tris Cl pH 7.4) using a polytron homogenizer. The homogenate was centrifuged at 10,000×g for 10 min at 4 degrees celsius and supernatant frozen at −80 degrees celsius prior to processing for leukotriene extractions. Supernatant samples were thawed and precipitated with final volume 10% ice cold methanol, held on ice for 30 minutes, then centrifuged at 10,000×g for 15 minutes. Denatured protein pellet discarded and lipid containing supernatant assayed for CysLTs (EIA method), at appropriate dilutions to be on the linear part of the standard curve using the procedure described in the Assay Designs kit with a sensitivity ˜30 pg CysLT/mL. FIG. 3 illustrates the increase in CysLTs in eye homogenates following ocular application of RSV in mice and the effect of Compound C on the levels of CysLTs following ocular application of RSV in mice. Topical treatment with Compound C reduced the levels of CysLTs in eye homogenates.

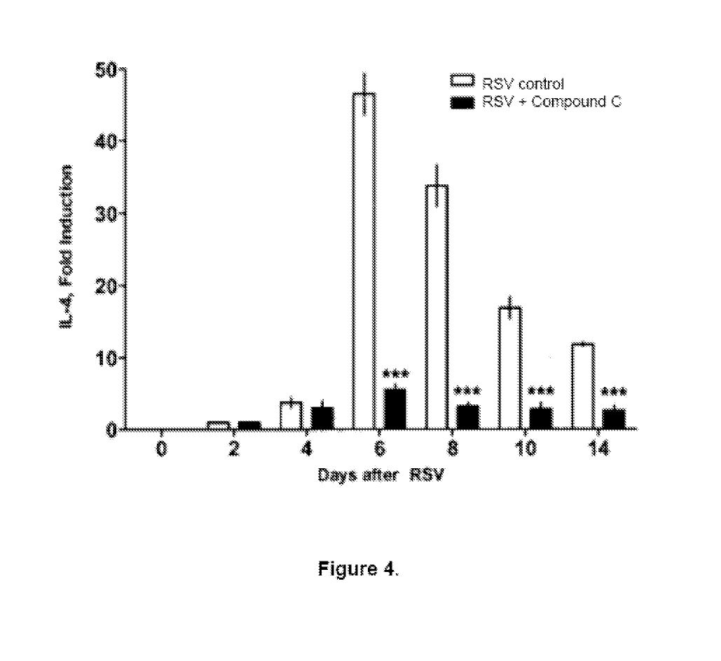

Interleukin-4 (IL-4) measurements. Eye extract as described above was used to determine levels of IL-4 in the extracts. FIG. 4 illustrates the increase in IL-4 in eye homogenates following ocular application of RSV in mice and the effect of a FLAP inhibitor on the levels of IL-4 following ocular application of RSV in mice. Topical treatment with Compound C reduced the levels of IL-4 in eye homogenates.

RSV RNA and protein: RSV is assayed in the ocular tissue on various days using both Western blot and viable virus assay (plaque forming unit; pfu) as described before. IL-5 is quantified by RT-PCR. RSV RNA and protein were measured in the eye and lung (Urnowey et al., BMC Microbiol. 6:26, 2006).

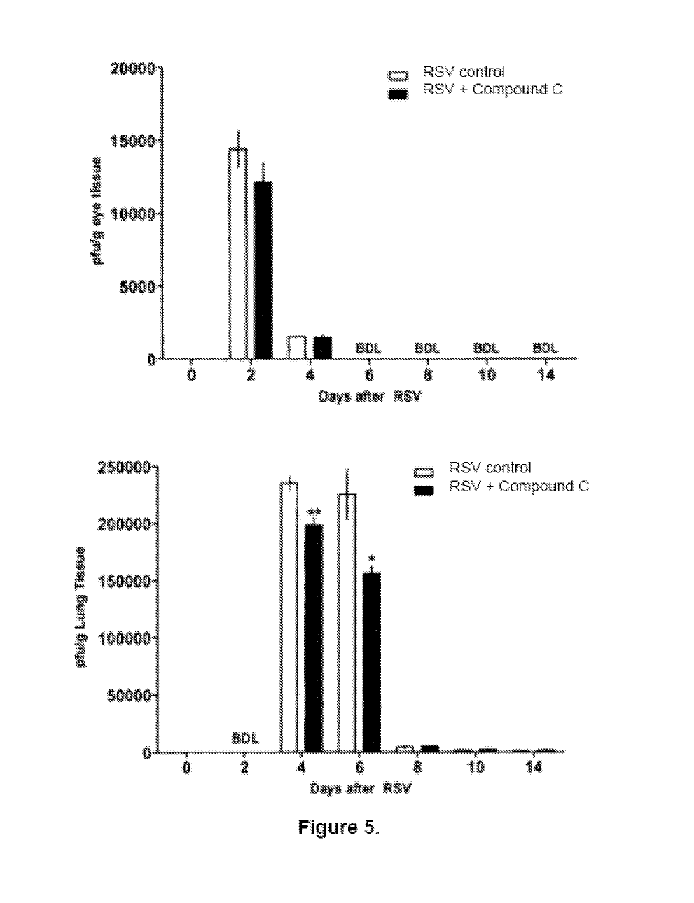

Administration of the FLAP inhibitor Compound C decreased the production of the Th2 cell cytokine IL-4 as described above but did not increase the viral load in the eye or the lung. Administration of the FLAP inhibitor compound C did not increase RSV mRNA or protein in the eye or the lung (FIG. 5). Compound C reduced RSV in the lung.

Infection of an eye eventually transmits into the lung, causing a standard respiratory infection. In some instances, eye serves as a portal of entry for RSV in to the lung. Thus, it is of interest to monitor the lung infection to determine if the ocular application of the FLAP inhibitor compound has any effect on virus replication and quantity of virus in the lung. The mice lung tissue is harvested, homogenates made and assayed for viral titer and Western blot (Matsuse H, et al. Allergology International 2007; 56:165-169; Bitko V, et al., J. Virol. 2007; 81(2):783-90.

Statistical Analysis. The pathology scores and ocular CysLT and/or IL-4 concentrations were subject to a two-way ANOVA followed by bonferroni post hoc analysis using GraphPad Prism software (GraphPad Software, San Diego, Calif.).

The examples and embodiments described herein are for illustrative purposes and various modifications or changes suggested to persons skilled in the art are to be included within the spirit and purview of this application and scope of the appended claims. The section headings used herein are for organizational purposes only and are not to be construed as limiting the subject matter described.

### Example 8

### Combination Therapy in a Mouse Subchronic Smoke Model

BALB/c mice were divided into groups and acclimatized in cages for 24 hours (day 0). The control group was exposed to air and the test group was exposed to smoke from seven unfiltered cigarettes per day for 8 days (day 1 to day 8). FLAP inhibitor compound (3-(3-(tert-butylthio)-1-(4-(6-methoxypyridin-3-yl)benzyl)-5-((5-methylpyridin-2-yl)methoxy)-1H-indol-2-yl)-2,2-dimethylpropanoic acid) (30 mg/kg, b.i.d.), DP₂ receptor antagonist 5-{2-[(N-benzyloxycarbonyl-N-ethyl-amino)-methyl]-4-trifluoromethyl-phenyl}-pyridin-3-yl)-acetic acid (10 mg/kg qd), or a combination of FLAP inhibitor compound (3-(3-(tert-butylthio)-1-(4-(6-methoxypyridin-3-yl)benzyl)-5-((5-methylpyridin-2-yl)methoxy)-1H-indol-2-yl)-2,2-dimethylpropanoic acid) (30 mg/kg, b.i.d.) and DP₂ receptor antagonist 5-{2-[(N-benzyloxycarbonyl-N-ethyl-amino)-methyl]-4-trifluoromethyl-phenyl}-pyridin-3-yl)-acetic acid (10 mg/kg qd) are administered starting at day 1 and up to day 13. On day 14, bronchoalveolar lavage fluid (BALF) is tested for influx of cells, cytokines, chemokines (e.g., KC, IL-17, MIP-2, IL-6), mucin, and/or proteins. Lung histology is also examined. Trough plasma concentration is shown in the following table:

| | Plasma concentration (nM) @ trough |
|---|---|
| DP₂ receptor antagonist | <10 |
| 10 mg/kg, qd | |
| FLAP inhibitor | 168 |
| 30 g/kg, b.i.d. | |
| DP₂ receptor antagonist + | <10 (DP₂ receptor |
| FLAP inhibitor | antagonist)/136 (FLAP inhibitor) |

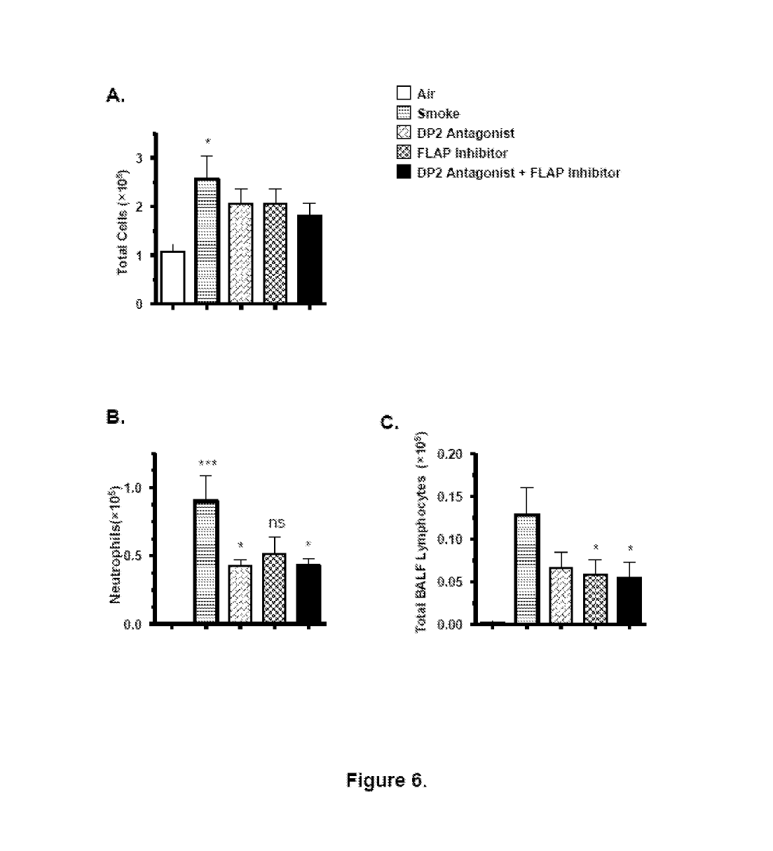

FIG. 6 illustrates the effect of FLAP inhibition, DP₂ receptor antagonism and combination of FLAP inhibition and DP₂ receptor antagonism on the number of total cells (FIG. 6A), neutrophils (FIG. 6B) and lymphocytes (FIG. 6C) present in BALF. In FIG. 6A, * represents P<0.05 vs. air, one-way ANOVA; in FIG. 6B, * is P<0.05 vs. smoke, one-way ANOVA with Tukey's, *** is P<0.001 vs. air, one-way ANOVA with Tukey's, “ns” is P=0.055, one-tailed t-test; and in FIG. 6C, * is P<0.05 vs. smoke, one-tailed t-test.

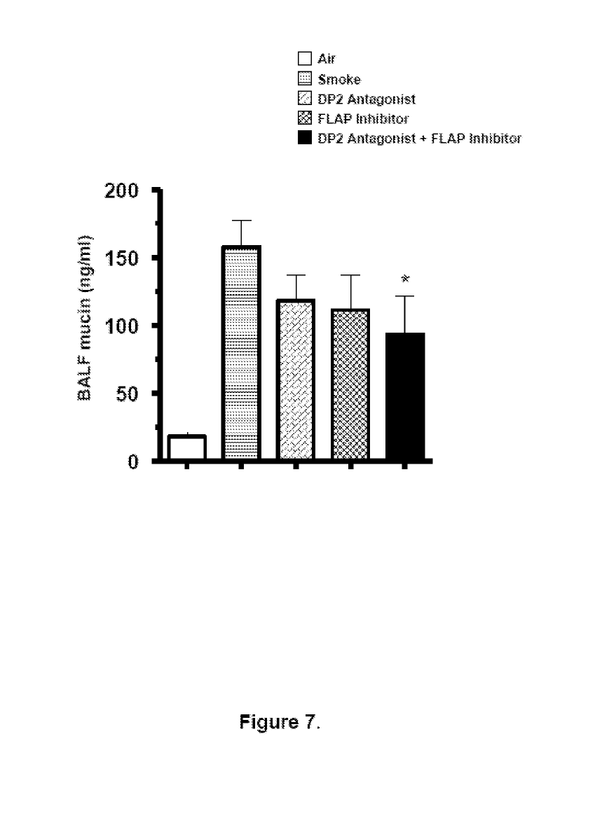

FIG. 7 illustrates the effect of a FLAP inhibitor, a DP₂ receptor antagonist and a combination of a FLAP inhibitor and a DP₂ receptor antagonist on the presence of mucin in BALF. In FIG. 7A, * represents P<0.05 vs. smoke, one-tailed t-test. In the subchronic smoking mouse model, the effects of a combination of a FLAP inhibitor compound and a DP₂ receptor antagonist compound on mucin secretion in BALF were additive, i.e., a combination of a FLAP inhibitor compound and a DP₂ receptor antagonist reduced the amount of mucin in BALF more than each compound alone. In one aspect, the effects of topical administration of a FLAP inhibitor compound, either alone or in combination with a DP₂ receptor antagonist, to an eye has the same effects (e.g., mechanistically expected) as observed in the BALF.

The examples and embodiments described herein are for illustrative purposes and various modifications or changes suggested to persons skilled in the art are to be included within the spirit and purview of this application and scope of the appended claims. The section headings used herein are for organizational purposes only and are not to be construed as limiting the subject matter described.

1. An ophthalmic formulation comprising a FLAP inhibitor compound in an amount effective for the treatment of an ophthalmic disease, disorder or condition in a mammal, and at least one suitable pharmaceutically acceptable excipient to provide a solution, suspension, ointment, cream, lotion, niosome, pharmacosome, ointment, or gel.

2. The ophthalmic formulation of claim 1, wherein the FLAP inhibitor compound inhibits leukotriene synthesis, antagonizes a leukotriene receptor, inhibits Interleukin-4 (IL-4) synthesis, or inhibits mucin synthesis.

3. The ophthalmic formulation of claim 1, wherein said ophthalmic disease, disorder or condition is age-related macular degeneration, allergic conjunctivitis, anterior segment scarring, blepharitis, blepharoconjunctivitis, a bullous disorder, cicatricial pemphigoid, conjunctival melanoma, conjunctivitis, contact lens-associated giant papillary conjunctivitis, diabetic retinopathy, dry eye, episcleritis, glaucoma, gliosis, granuloma annulare, Graves' ophthalmopathy, intraocular melanoma, keratitis, keratoconjunctivitis, pain, pinguecula, post-surgical pain, proliferative vitreoretinopathy, pterygia, scarring, scleritis, viral infection, Sjögren's syndrome, uveitis, vernal keratoconjunctivitis or combinations thereof.

4. The ophthalmic formulation of claim 1, wherein the FLAP inhibitor compound is a compound of Formula (I), or a pharmaceutically acceptable salt, or N-oxide thereof: 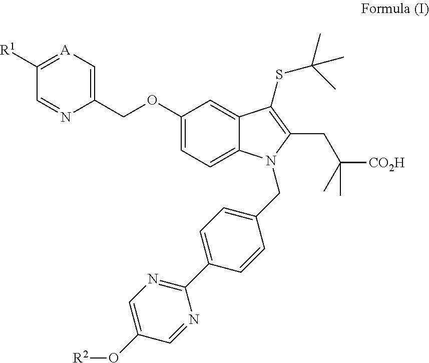
wherein,
A is CH or N;
R¹ is H, —F, —Cl, —Br, —CN, C₁-C₄alkyl, C₁-C₄-fluoroalkyl, —O—C₁-C₄alkyl, or —O—C₁-C₄-fluoroalkyl;
R² is C₁-C₄alkyl or C₁-C₄-fluoroalkyl.

5. The ophthalmic formulation of claim 4, wherein the FLAP inhibitor compound is 3-[3-tert-Butylsulfanyl-1-[4-(5-methoxy-pyrimidin-2-yl)-benzyl]-5-(5-methyl-pyridin-2-ylmethoxy)-1H-indol-2-yl]-2,2-dimethyl-propionic acid (Compound A); or 3-[3-tert-Butylsulfanyl-1-[4-(5-methoxy-pyrimidin-2-yl)-benzyl]-5-(5-methyl-pyrazin-2-ylmethoxy)-1H-indol-2-yl]-2,2-dimethyl-propionic acid (Compound B), or a pharmaceutically acceptable salt, or N-oxide thereof.

6. The ophthalmic formulation of claim 1, wherein the FLAP inhibitor compound is a compound of Formula (II), or a pharmaceutically acceptable salt, or N oxide thereof: 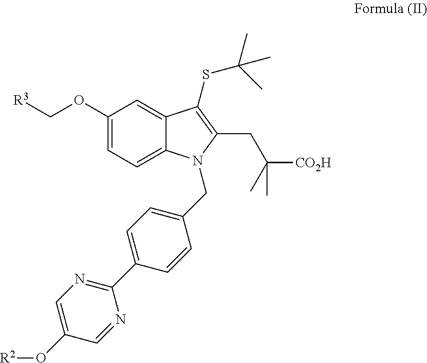
wherein,
R² is C₁-C₄alkyl or C₁-C₄-fluoroalkyl;
R³ is a substituted or unsubstituted monocyclic or bicyclic heterocycloalkyl.

7. The ophthalmic formulation of claim 6, wherein R³ is selected from the group consisting of: 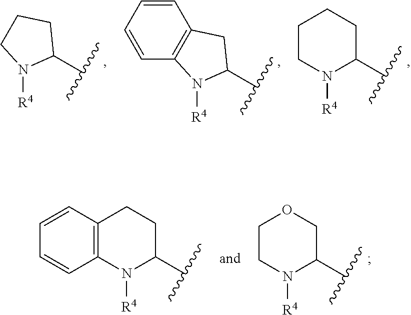
R⁴ is H, —C(═O)R⁵ or —SO₂—C₁-C₄alkyl; R⁵ is C₁-C₄alkyl, C₁-C₄-fluoroalkyl, substituted or unsubstituted phenyl, substituted or unsubstituted heteroaryl, or —O—C₁-C₄alkyl.

8. The ophthalmic formulation of claim 7, wherein the compound of Formula (II) has the following structure:

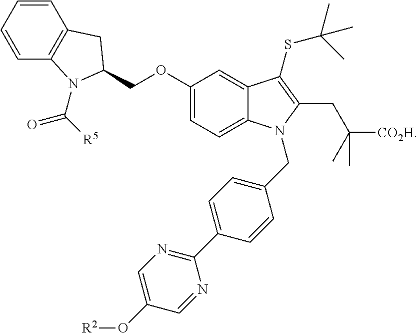

9. The ophthalmic formulation of claim 8, wherein the FLAP inhibitor compound is 3-{5-((S)-1-Acetyl-2,3-dihydro-1H-indol-2-ylmethoxy)-3-tert-butylsulfanyl-1-[4-(5-methoxy-pyrimidin-2-yl)-benzyl]-1H-indol-2-yl}-2,2-dimethyl-propionic acid (Compound C), 3-{5-((S)-1-Acetyl-2,3-dihydro-1H-indol-2-ylmethoxy)-3-tert-butylsulfanyl-1-[4-(5-ethoxy-pyrimidin-2-yl)-benzyl]-1H-indol-2-yl}-2,2-dimethyl-propionic acid (Compound K), or a pharmaceutically acceptable salt, or N-oxide thereof.

10. The topical formulation of claim 1, wherein the FLAP inhibitor compound is
3-[3-tert-butylsulfanyl-1-(4-chloro-benzyl)-5-isopropyl-1H-indol-2-yl]-2,2-dimethyl-propionic acid (MK886); 3-[3-tert-butylsulfanyl-1-(4-chloro-benzyl)-5-(quinolin-2-ylmethoxy)-1H-indol-2-yl]-2,2-dimethyl-propionic acid (MK591); cyclopentyl-[4-(quinolin-2-ylmethoxy)-phenyl]-acetic acid (DG031; BAY X1005); 3-[3-tert-Butylsulfanyl-1-[4-(5-methoxy-pyrimidin-2-yl)-benzyl]-5-(5-methyl-pyridin-2-ylmethoxy)-1H-indol-2-yl]-2,2-dimethyl-propionic acid (Compound A); 3-[3-tert-Butylsulfanyl-1-[4-(5-methoxy-pyrimidin-2-yl)-benzyl]-5-(5-methyl-pyrazin-2-ylmethoxy)-1H-indol-2-yl]-2,2-dimethyl-propionic acid (Compound B); 3-{5-((S)-1-Acetyl-2,3-dihydro-1H-indol-2-ylmethoxy)-3-tert-butylsulfanyl-1-[4-(5-methoxy-pyrimidin-2-yl)-benzyl]-1H-indol-2-yl}-2,2-dimethyl-propionic acid (Compound C); 3-[3-tert-Butylsulfanyl-1-[4-(6-methoxy-pyridin-3-yl)-benzyl]-5-(pyridin-2-ylmethoxy)-1H-indol-2-yl]-2,2-dimethyl-propionic acid (Compound D); 3-[3-tert-Butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid (Compound E); 3-[3-tert-Butylsulfanyl-1-[4-(5-fluoro-pyridin-2-yl)-benzyl]-5-(quinolin-2-ylmethoxy)-1H-indol-2-yl]-2,2-dimethyl-propionic acid (Compound F); 2-[3-tert-Butylsulfanyl-1-[4-(5-methoxy-pyrimidin-2-yl)-benzyl]-5-(5-methyl-pyridin-2-ylmethoxy)-1H-indol-2-ylmethyl]-2-ethyl-butyric acid (Compound G); 3-[3-tert-Butylsulfanyl-1-[4-(6-methoxy-pyridin-3-yl)-benzyl]-5-(5-methyl-pyridin-2-ylmethoxy)-1H-indol-2-yl]-2,2-dimethyl-propionic acid (Compound H); 3-[5-((S)-1-Acetyl-pyrrolidin-2-ylmethoxy)-3-tert-butylsulfanyl-1-(4-chloro-benzyl)-1H-indol-2-yl]-2,2-dimethyl-propionic acid (Compound I); 3-[3-tert-butylsulfanyl-1-[4-(5-fluoro-pyridin-2-yl)-benzyl]-5-(pyridin-2-ylmethoxy)-1H-indol-2-yl]-2,2-dimethyl-propionic acid (Compound J); 3-{5-((S)-1-Acetyl-2,3-dihydro-1H-indol-2-ylmethoxy)-3-tert-butylsulfanyl-1-[4-(5-ethoxy-pyrimidin-2-yl)-benzyl]-1H-indol-2-yl}-2,2-dimethyl-propionic acid (Compound K); or a pharmaceutically acceptable salt, or N-oxide thereof.

11. The ophthalmic formulation of claim 1, further comprising a therapeutically-effective amount of an second compound, wherein the second compound is an antibiotic; anti-fungal agent; steroid anti-inflammatory agent; non-steroidal anti-inflammatory agent; antihistamine; antiviral; alpha agonist; beta blocker; carbonic anhydrase inhibitor; miotic; prostaglandin; anti-angiogenesis agent; loteprednol etabonate, mast cell stabilizer, cyclosporine, or DP2 antagonist.

12. A method of treating of an ophthalmic disease, disorder or condition in a mammal comprising administering an ophthalmic formulation comprising a therapeutically-effective amount of a FLAP inhibitor compound to at least one eye of the mammal with an ophthalmic disorder.

13. The method of claim 12, wherein the FLAP inhibitor compound inhibits leukotriene synthesis, antagonizes a leukotriene receptor, inhibits Interleukin-4 (IL-4) synthesis, or inhibits mucin synthesis.

14. The method of claims 12, wherein the ophthalmic formulation is in the form of a solution, a suspension, an ointment, a gel, a cream, a liposome, a niosome, a pharmacosome, a nanoparticle, or combinations thereof.

15. The method of claim 12, wherein the ophthalmic formulation is administered via implantation, insertion, injection, spraying, washing, or combinations thereof.

16. The method of claim 12, wherein the ophthalmic disease, disorder or condition is a age-related macular degeneration, allergic conjunctivitis, anterior segment scarring, blepharitis, blepharoconjunctivitis, a bullous disorder, cicatricial pemphigoid, conjunctival melanoma, conjunctivitis, contact lens-associated giant papillary conjunctivitis, diabetic retinopathy, dry eye, episcleritis, glaucoma, gliosis, granuloma annulare, Graves' ophthalmopathy, intraocular melanoma, keratitis, keratoconjunctivitis, pain, pinguecula, post-surgical pain, proliferative vitreoretinopathy, pterygia, scarring, scleritis, Sjögren's syndrome, uveitis, vernal keratoconjunctivitis, post-surgical inflammation, post-surgical scarring or combinations thereof.

17. The method of claim 12, wherein the FLAP inhibitor compound is a compound of Formula (I), or a pharmaceutically acceptable salt, or N-oxide thereof: 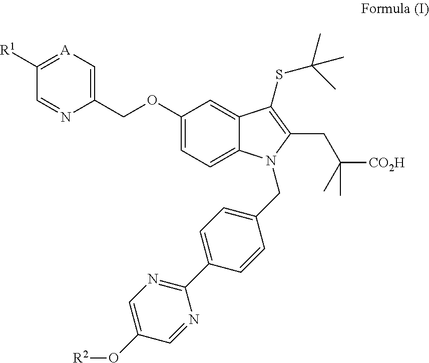
wherein,
A is CH or N;
R¹ is H, —F, —Cl, —Br, —CN, C₁-C₄alkyl, C₁-C₄-fluoroalkyl, —O—C₁-C₄alkyl, or —O—C₁-C₄-fluoroalkyl;
R² is C₁-C₄alkyl or C₁-C₄-fluoroalkyl.

18. The method of claim 17, wherein the FLAP inhibitor compound is 3-[3-tert-Butylsulfanyl-1-[4-(5-methoxy-pyrimidin-2-yl)-benzyl]-5-(5-methyl-pyridin-2-ylmethoxy)-1H-indol-2-yl]-2,2-dimethyl-propionic acid (Compound A); or 3-[3-tert-Butylsulfanyl-1-[4-(5-methoxy-pyrimidin-2-yl)-benzyl]-5-(5-methyl-pyrazin-2-ylmethoxy)-1H-indol-2-yl]-2,2-dimethyl-propionic acid (Compound B), or a pharmaceutically acceptable salt, or N-oxide thereof.

19. The method of claim 12, wherein the FLAP inhibitor compound is a compound of Formula (II), or a pharmaceutically acceptable salt, or N-oxide thereof: 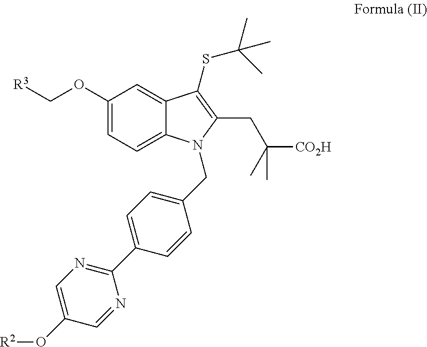
wherein,
R² is C₁-C₄alkyl or C₁-C₄-fluoroalkyl;
R³ is a substituted or unsubstituted monocyclic or bicyclic heterocycloalkyl.

20. The method of claim 19, wherein R³ is selected from the group consisting of: 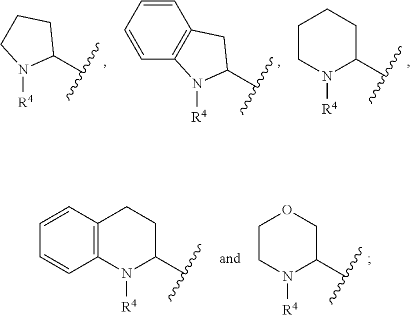
R⁴ is H, —C(═O)R⁵ or —SO₂—C₁-C₄alkyl; R⁵ is C₁-C₄alkyl, C₁-C₄-fluoroalkyl, substituted or unsubstituted phenyl, substituted or unsubstituted heteroaryl, or —O—C₁-C₄alkyl.

21. The method of claim 20, wherein the compound of Formula (II) has the following structure:

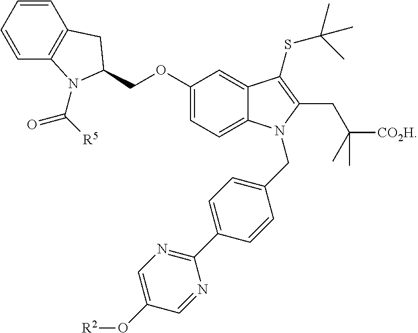

22. The method of claim 21, wherein the FLAP inhibitor compound is 3-{5-((S)-1-Acetyl-2,3-dihydro-1H-indol-2-ylmethoxy)-3-tert-butylsulfanyl-1-[4-(5-methoxy-pyrimidin-2-yl)-benzyl]-1H-indol-2-yl}-2,2-dimethyl-propionic acid (Compound C), 3-{5-((S)-1-Acetyl-2,3-dihydro-1H-indol-2-ylmethoxy)-3-tert-butylsulfanyl-1-[4-(5-ethoxy-pyrimidin-2-yl)-benzyl]-1H-indol-2-yl}-2,2-dimethyl-propionic acid (Compound K), or a pharmaceutically acceptable salt, or N-oxide thereof.

23. The method of claim 12, wherein the FLAP inhibitor compound is selected from: 3-[3-tert-butylsulfanyl-1-(4-chloro-benzyl)-5-isopropyl-1H-indol-2-yl]-2,2-dimethyl-propionic acid (MK886); 3-[3-tert-butylsulfanyl-1-(4-chloro-benzyl)-5-(quinolin-2-ylmethoxy)-1H-indol-2-yl]-2,2-dimethyl-propionic acid (MK591); cyclopentyl-[4-(quinolin-2-ylmethoxy)-phenyl]-acetic acid (DG031; BAY X1005); 3-[3-tert-Butylsulfanyl-1-[4-(5-methoxy-pyrimidin-2-yl)-benzyl]-5-(5-methyl-pyridin-2-ylmethoxy)-1H-indol-2-yl]-2,2-dimethyl-propionic acid (Compound A); 3-[3-tert-Butylsulfanyl-1-[4-(5-methoxy-pyrimidin-2-yl)-benzyl]-5-(5-methyl-pyrazin-2-ylmethoxy)-1H-indol-2-yl]-2,2-dimethyl-propionic acid (Compound B); 3-{5-((S)-1-Acetyl-2,3-dihydro-1H-indol-2-ylmethoxy)-3-tert-butylsulfanyl-1-[4-(5-methoxy-pyrimidin-2-yl)-benzyl]-1H-indol-2-yl}-2,2-dimethyl-propionic acid (Compound C); 3-[3-tert-Butylsulfanyl-1-[4-(6-methoxy-pyridin-3-yl)-benzyl]-5-(pyridin-2-ylmethoxy)-1H-indol-2-yl]-2,2-dimethyl-propionic acid (Compound D); 3-[3-tert-Butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid (Compound E); 3-[3-tert-Butylsulfanyl-1-[4-(5-fluoro-pyridin-2-yl)-benzyl]-5-(quinolin-2-ylmethoxy)-1H-indol-2-yl]-2,2-dimethyl-propionic acid (Compound F); 2-[3-tert-Butylsulfanyl-1-[4-(5-methoxy-pyrimidin-2-yl)-benzyl]-5-(5-methyl-pyridin-2-ylmethoxy)-1H-indol-2-ylmethyl]-2-ethyl-butyric acid (Compound G); 3-[3-tert-Butylsulfanyl-1-[4-(6-methoxy-pyridin-3-yl)-benzyl]-5-(5-methyl-pyridin-2-ylmethoxy)-1H-indol-2-yl]-2,2-dimethyl-propionic acid (Compound H); 3-[5-((S)-1-Acetyl-pyrrolidin-2-ylmethoxy)-3-tert-butylsulfanyl-1-(4-chloro-benzyl)-1H-indol-2-yl]-2,2-dimethyl-propionic acid (Compound I); 3-[3-tert-butylsulfanyl-1-[4-(5-fluoro-pyridin-2-yl)-benzyl]-5-(pyridin-2-ylmethoxy)-1H-indol-2-yl]-2,2-dimethyl-propionic acid (Compound J); 3-{5-((S)-1-Acetyl-2,3-dihydro-1H-indol-2-ylmethoxy)-3-tert-butylsulfanyl-1-[4-(5-ethoxy-pyrimidin-2-yl)-benzyl]-1H-indol-2-yl}-2,2-dimethyl-propionic acid (Compound K); or a pharmaceutically acceptable salt, or N-oxide thereof.

24. The method of claim 12, further comprising administering to the mammal a therapeutically-effective amount of a compound selected from antibiotics; anti-fungal agents; steroid anti-inflammatory agents; non-steroidal anti-inflammatory agents; antihistamines; antivirals; alpha agonists; beta blockers; carbonic anhydrase inhibitors; miotics; prostaglandins; anti-angiogenesis agents; loteprednol etabonate, mast cell stabilizers, cyclosporine, and DP2 antagonists.

25. A compound of Formula (I), or a pharmaceutically acceptable salt, or N-oxide thereof: 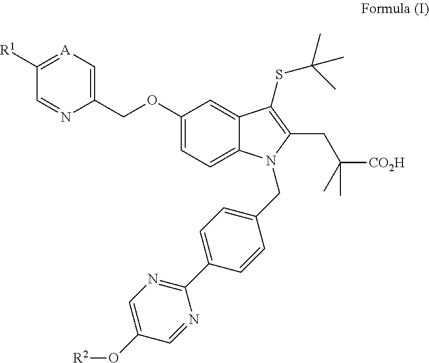
wherein,
A is CH or N;
R¹ is —F, —Cl, —Br, —CN, C₁-C₄alkyl, C₁-C₄-fluoroalkyl, —O—C₁-C₄alkyl, or —O—C₁-C₄-fluoroalkyl;
R² is C₁-C₄alkyl or C₁-C₄-fluoroalkyl.

26. The compound of claim 25, wherein the compound of Formula (I) is 3-[3-tert-Butylsulfanyl-1-[4-(5-methoxy-pyrimidin-2-yl)-benzyl]-5-(5-methyl-pyridin-2-ylmethoxy)-1H-indol-2-yl]-2,2-dimethyl-propionic acid (Compound A); or 3-[3-tert-Butylsulfanyl-1-[4-(5-methoxy-pyrimidin-2-yl)-benzyl]-5-(5-methyl-pyrazin-2-ylmethoxy)-1H-indol-2-yl]-2,2-dimethyl-propionic acid (Compound B), or a pharmaceutically acceptable salt, or N-oxide thereof.

27. A compound of Formula (II), or a pharmaceutically acceptable salt, or N-oxide thereof: 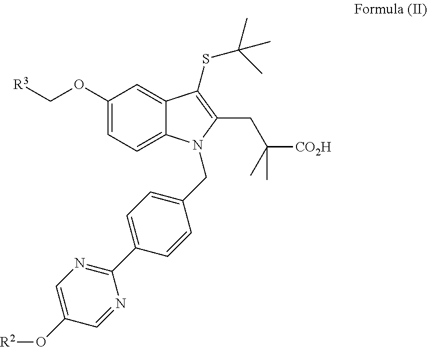
wherein,
R² is C₁-C₄alkyl or C₁-C₄-fluoroalkyl;
R³ is a substituted or unsubstituted monocyclic or bicyclic heterocycloalkyl.

28. The compound of claim 27, or a pharmaceutically acceptable salt, or N-oxide thereof, wherein R³ is selected from the group consisting of: 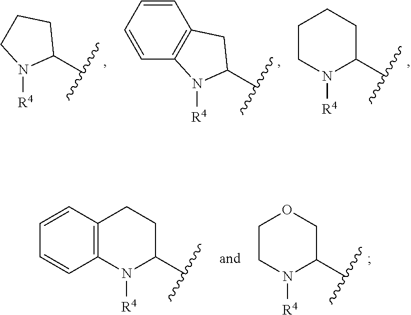
R⁴ is H, —C(═O)R⁵ or —SO₂—C₁-C₄alkyl; R⁵ is C₁-C₄alkyl, C₁-C₄-fluoroalkyl, substituted or unsubstituted phenyl, substituted or unsubstituted heteroaryl, or —O—C₁-C₄alkyl.

29. The compound of claim 28, or a pharmaceutically acceptable salt, or N-oxide thereof, wherein the compound of Formula (II) has the following structure:

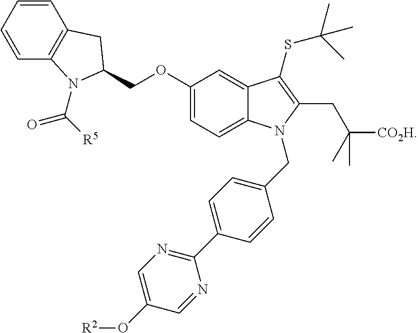

30. The compound of claim 29, wherein the compound is 3-{5-((S)-1-Acetyl-2,3-dihydro-1H-indol-2-ylmethoxy)-3-tert-butylsulfanyl-1-[4-(5-methoxy-pyrimidin-2-yl)-benzyl]-1H-indol-2-yl}-2,2-dimethyl-propionic acid (Compound C), or 3-{5-((S)-1-Acetyl-2,3-dihydro-1H-indol-2-ylmethoxy)-3-tert-butylsulfanyl-1-[4-(5-ethoxy-pyrimidin-2-yl)-benzyl]-1H-indol-2-yl}-2,2-dimethyl-propionic acid (Compound K), or a pharmaceutically acceptable salt, or N-oxide thereof.
